(19) United States
(12) Patent Application Publication
(10) Pub. No.: US 2026/0297207 A1
(43) Pub. Date: Oct. 1, 2026

(54) ANTI-TL1A×IL23 BISPECIFIC ANTIBODIES AND THEIR APPLICATIONS THEREOF

(71) Applicant: Simcere Pharmaceutical Co., Ltd., Nanjing (CN)

(72) Inventors: Xiaofeng Zhao, Nanjing (CN); Xiaoqing Liu, Nanjing (CN); Fudong Wang, Nanjing (CN); Yuxi Yan, Nanjing (CN)

(73) Assignee: Simcere Pharmaceutical Co., Ltd., Nanjing (CN)

(21) Appl. No.: 19/707,211

(22) Filed: Jun. 12, 2026

Related U.S. Application Data
(63) Continuation of application No. 19/632,481, filed on Mar. 30, 2026.

(30) Foreign Application Priority Data
Mar. 31, 2025 (CN) 202510397834.9
Mar. 6, 2026 (CN) 202610274906.5

(51) Int. Cl. C07K 16/28; A61P 37/06; C07K 16/24

(52) U.S. Cl. CPC C07K 16/2875; A61P 37/06; C07K 16/244; C07K 2317/31; C07K 2317/569

(57) ABSTRACT

The present disclosure relates to an anti-TL1A×IL23 bispecific molecule and use thereof. Specifically, disclosed are a bispecific molecule specifically binding to human TNF-like ligand 1A (TL1A) and interleukin 23, and corresponding encoding nucleic acid molecules, vectors, cells, preparation methods, pharmaceutical compositions, pharmaceutical use, and treatment methods for diseases, which are of great significance for the development of a drug for treating an immune-mediated inflammatory disease (IMID) (e.g., autoimmune diseases and inflammatory diseases).

### RELATED APPLICATIONS

This application is a Continuation of U.S. application Ser. No. 19/632,481, filed Mar. 30, 2026, entitled “ANTI-TL1A×IL23 BISPECIFIC ANTIBODIES AND THEIR APPLICATIONS THEREOF”. Foreign priority benefits are claimed under 35 U.S.C. § 119(a)-(d) or 35 U.S.C. § 365(b) of Chinese application number 202610274906.5, filed Mar. 6, 2026 and Chinese application number 202510397834.9, filed Mar. 31, 2025. The entire contents of these applications are incorporated herein by reference in their entirety.

### REFERENCE TO AN ELECTRONIC SEQUENCE LISTING

The contents of the electronic sequence listing (B169470016US01-SEQ-GJM.xml; Size: 81,034 bytes; and Date of Creation: Jun. 12, 2026) are herein incorporated by reference in their entirety.

### TECHNICAL FIELD

The present disclosure relates to the field of medicine, and particularly relates to a bispecific molecule capable of specifically binding to human TNF-like ligand 1A (TL1A) and interleukin 23 (IL23), a preparation method therefor, a pharmaceutical composition thereof, and use thereof in treating an autoimmune disease or inflammatory disease.

### BACKGROUND

Inflammatory bowel disease (IBD), including Crohn's disease (CD) and ulcerative colitis (UC), is an autoimmune disease. Common symptoms include diarrhea and hematochezia, and long-term inflammation may affect organs throughout the body. As a chronic non-specific inflammatory bowel disease, IBD is characterized by recurrent episodes. Currently, there is no method for its complete cure, and it imposes a heavy global disease burden, with its incidence and prevalence continuing to rise. Traditional therapeutic drugs for IBD include glucocorticoids, antibiotics, immunosuppressants, aminosalicylic acid, and the like. Following the approval of infliximab and adalimumab targeting TNF-α for UC/CD indications, IBD also entered the “biological formulation” era. However, there is still a significant proportion of the target patient population that does not respond or will lose response over time to existing biological agents, and there is still a significant unmet clinical need.

Tumor necrosis factor-like cytokine 1A (TL1A) is a member of the tumor necrosis factor family. It is expressed in different immune cells, such as monocytes, macrophages, dendritic cells, T cells, and non-immune cells, e.g., synovial fibroblasts and endothelial cells. The basal level of TL1A is low, but rises rapidly after immune activation, and studies have shown that the expression level of TL1A in the colonic tissue of patients with IBD is correlated with the severity of inflammation.

Interleukin-23 (IL-23) is a heterodimeric cytokine composed of two subunits, p19 and p40. p40 is shared with IL-12 and is primarily produced by activated dendritic cells, macrophages, monocytes, etc. The IL23 receptor includes 2 subunits: the IL-12 receptor β1 and the IL23 receptor. By interacting with the IL-23 receptor expressed on the surface of T cells, NK cells, mononuclear macrophages, or dendritic cells, IL-23 activates downstream signaling pathways to exert its biological functions. Both preclinical and clinical studies have confirmed that IL23 inhibition can provide efficacy comparable to that of anti-IL-12P40 inhibition. Simultaneously, neutralizing IL-23 without inhibiting the IL-12 pathway reduces the risk of infection.

### SUMMARY

Studies have shown that DR3 and IL23R are co-expressed on inflammatory immune cells such as CD4⁺ T cells, CD8⁺ T cells, and ILC cells in biological specimens of IBD patients, and targeting a variety of disease factors can be achieved, for example, by co-administration or combined use of two separate biological formulations. From practical and commercial perspectives, however, co-administration or combination still presents challenges. For instance, administering two separate injections may negatively impact patient compliance, and production of two separate drugs may increase overall costs. Therefore, bispecific antibodies capable of simultaneously binding to two different antigens can serve as a strategy to address the limitations associated with co-administration or combined use in the prior art.

In a first aspect, the present disclosure provides a molecule specifically binding to TL1A and IL23, and the molecule comprises an antibody or an antigen-binding fragment specifically binding to TL1A and IL23.

In some embodiments, the molecule comprises a first antigen-binding domain ABD1 and a second antigen-binding domain ABD2.

In some embodiments, the ABD1 and the ABD2 specifically bind to TL1A and IL23, respectively.

In some embodiments, the molecule is a bispecific molecule capable of specifically binding to TL1A and IL23.

In some embodiments, the ABD1 and the ABD2 are each independently selected from an antibody, an antibody fragment, a F(ab′)2, a Fab′, a Fab, an Fv, an scFv, a nanobody, and VHH.

In some embodiments, the first antigen-binding domain ABD1 comprises a heavy chain variable region VH1 and/or a light chain variable region VL1, and the second antigen-binding domain ABD2 comprises a heavy chain variable region VH2 and/or a light chain variable region VL2.

In some embodiments, the molecule further comprises a heavy chain constant region and/or a light chain constant region, wherein optionally, the heavy chain constant region and/or the light chain constant region are selected from an intact constant region sequence and a fragment thereof, wherein the constant region fragment comprises a CH1, a hinge region, a CH2, a CH3, a CL, or an Fc region; optionally, the heavy chain constant region is selected from human or murine IgG1, IgG2, IgG3, and IgG4 constant regions, and the light chain constant region is selected from a human or murine kappa constant region and a lambda constant region.

In some specific embodiments, the molecule comprises an IgG antibody and an scFv structure, wherein the scFv structure can be located at the N-terminus or C-terminus of a heavy chain or a light chain of the IgG antibody.

In some specific embodiments, the molecule comprises an IgG antibody and a VHH structure, wherein the VHH structure can be located at the N-terminus or C-terminus of a heavy chain or a light chain of the IgG antibody.

In some specific embodiments, the molecule is a “Crossmab” type antibody.

In some specific embodiments, the molecule comprises a Fab, an Fc region, and a VHH structure, wherein the VHH structure can be located on either of the two chains of the Fc region.

In some specific embodiments, (1) the first heavy chain and the second heavy chain are identical and have a structure as shown below:

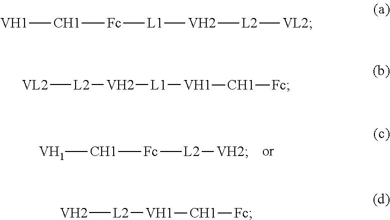

and/or (2) a first light chain and a second light chain of the molecule are identical and have the following structure: VL1-CL,
wherein the VH1/VH2 and/or the VL1/VL2 are optionally heavy chain variable regions and/or light chain variable regions specifically binding to TL1A or IL23, and the Fc is an Fc region of any one of the antibodies; the two light chains comprise VL1-CL; the L1 and the L2 are identical or different linkers.

Preferably, the L1 is (G4S) 4 (SEQ ID NO: 31), and the L2 is (G4S)₃ (SEQ ID NO: 32).

In some specific embodiments, the molecule comprises a first heavy chain and a second heavy chain, as well as a first light chain and a second light chain, wherein the first heavy chain comprises VH1-CH1-Fc, the second heavy chain comprises VH2-CL-Fc, the first light chain comprises VL1-CL, and the second light chain comprises VL2-CH1.

In some specific embodiments, the molecule comprises three polypeptide chains: a first heavy chain, a second heavy chain, and one light chain, wherein (1) the first heavy chain and the second heavy chain have a structure as shown below:
(a) the first heavy chain VH1-CH1-Fc and the second heavy chain VH2-Fc;
(b) the first heavy chain VH1-CH1-Fc-VH2 and the second heavy chain Fc; or
(c) the first heavy chain VH1-CH1-Fc and the second heavy chain Fc-VH2;
and (2) the light chain has the following structure: VL1-CL.

The VH1/VH2 and/or the VL1/VL2 are optionally heavy chain variable regions and/or light chain variable regions specifically binding to TL1A or IL23, and the Fc is an Fc region of any one of the antibodies.

In some embodiments, the antigen-binding domain specifically binding to TL1A in the molecule comprises a heavy chain variable region and/or a light chain variable region of the antibody.

In some specific embodiments, (a) the heavy chain variable region comprises an HCDR1, an HCDR2, and an HCDR3, wherein the HCDR1, the HCDR2, and the HCDR3 have the HCDR1, the HCDR2, and the HCDR3 of a sequence set forth in any one of SEQ ID NO: 9, 11, 13, 15, 69, or 70, or sequences having at least 70% identity or at most 3 mutations compared thereto; and/or (b) the light chain variable region comprises an LCDR1, an LCDR2, and an LCDR3, wherein the LCDR1, the LCDR2, and the LCDR3 have the LCDR1, the LCDR2, and the LCDR3 of a sequence set forth in any one of SEQ ID NO: 10, 12, or 14, or sequences having at least 70% identity or at most 3 mutations compared thereto; preferably, the HCDR1-3 and/or the LCDR1-3 are selected from Table 7.

In some specific embodiments, (a) the heavy chain variable region comprises an HCDR1, an HCDR2, and an HCDR3, wherein the HCDR1, the HCDR2, and the HCDR3 have sequences set forth in SEQ ID NOs: 36-38, SEQ ID NOs: 42-44, SEQ ID NOs: 48-50, SEQ ID NOs: 51-53, SEQ ID NOs: 75-77, or SEQ ID NOs: 78-80, or sequences having at least 70% identity or at most 3 mutations compared thereto; and/or (b) the light chain variable region comprises an LCDR1, an LCDR2, and an LCDR3, wherein the LCDR1, the LCDR2, and the LCDR3 have sequences set forth in SEQ ID NOs: 33-35, SEQ ID NOs: 39-41, or SEQ ID NOs: 45-47, or sequences having at least 70% identity or at most 3 mutations compared thereto.

In some embodiments, the antigen-binding domain specifically binding to TL1A in the molecule comprises:
(1) an HCDR1, an HCDR2, and an HCDR3 of the sequence set forth in SEQ ID NO: 9, and an LCDR1, an LCDR2, and an LCDR3 of the sequence set forth in SEQ ID NO: 10, or sequences having at least 70% identity or at most 3 mutations compared thereto; or
(2) an HCDR1, an HCDR2, and an HCDR3 of the sequence set forth in SEQ ID NO: 11, and an LCDR1, an LCDR2, and an LCDR3 of the sequence set forth in SEQ ID NO: 12, or sequences having at least 70% identity or at most 3 mutations compared thereto; or
(3) an HCDR1, an HCDR2, and an HCDR3 of the sequence set forth in SEQ ID NO: 13, and an LCDR1, an LCDR2, and an LCDR3 of the sequence set forth in SEQ ID NO: 14, or sequences having at least 70% identity or at most 3 mutations compared thereto; or
(4) an HCDR1, an HCDR2, and an HCDR3 of the sequence set forth in SEQ ID NO: 15, or sequences having at least 70% identity or at most 3 mutations compared thereto; or
(5) an HCDR1, an HCDR2, and an HCDR3 of the sequence set forth in SEQ ID NO: 69, or sequences having at least 70% identity or at most 3 mutations compared thereto; or
(6) an HCDR1, an HCDR2, and an HCDR3 of the sequence set forth in SEQ ID NO: 70, or sequences having at least 70% identity or at most 3 mutations compared thereto.

Preferably, the HCDR1-3 and/or the LCDR1-3 are determined according to the Kabat, Chothia, or IMGT scheme.

In some embodiments, the antigen-binding domain specifically binding to TL1A in the molecule comprises a heavy chain variable region and/or a light chain variable region of the antibody.

In some specific embodiments, the heavy chain variable region has the sequence set forth in any one of SEQ ID NO: 9, 11, 13, 15, 69, or 70, or a sequence having at least 70% identity or at most 15 mutations compared thereto; and/or
the light chain variable region has the sequence set forth in any one of SEQ ID NO: 10, 12, or 14, or a sequence having at least 70% identity or at most 15 mutations compared thereto.

In some embodiments, the antigen-binding domain specifically binding to IL23 in the molecule comprises a heavy chain variable region and/or a light chain variable region of the antibody.

In some specific embodiments, (a) the heavy chain variable region comprises an HCDR1, an HCDR2, and an HCDR3, wherein the HCDR1, the HCDR2, and the HCDR3 have the HCDR1, the HCDR2, and the HCDR3 of a sequence set forth in any one of SEQ ID NO: 16, 18, 20, 71, or 73, or sequences having at least 70% identity or at most 3 mutations compared thereto; and/or (b) the light chain variable region comprises an LCDR1, an LCDR2, and an LCDR3, wherein the LCDR1, the LCDR2, and the LCDR3 have the LCDR1, the LCDR2, and the LCDR3 of a sequence set forth in any one of SEQ ID NO: 17, 19, 72, or 74, or sequences having at least 70% identity or at most 3 mutations compared thereto; preferably, the HCDR1-3 and/or the LCDR1-3 are selected from Table 7.

In some specific embodiments, (a) the heavy chain variable region comprises an HCDR1, an HCDR2, and an HCDR3, wherein the HCDR1, the HCDR2, and the HCDR3 have sequences set forth in SEQ ID NOs: 54-56, SEQ ID NOs: 60-62, SEQ ID NOs: 66-68, SEQ ID NOs: 54, 81, and 56, or SEQ ID NOs: 54, 84, and 56, or sequences having at least 70% identity or at most 3 mutations compared thereto; and/or (b) the light chain variable region comprises an LCDR1, an LCDR2, and an LCDR3, wherein the LCDR1, the LCDR2, and the LCDR3 have sequences set forth in SEQ ID NOs: 57-59, SEQ ID NOs: 63-65, SEQ ID NOs: 82, 83, and 59, or SEQ ID NOs: 85, 58, and 59, or sequences having at least 70% identity or at most 3 mutations compared thereto.

In some embodiments, the antigen-binding domain specifically binding to IL23 in the molecule comprises:
(1) an HCDR1, an HCDR2, and an HCDR3 of the sequence set forth in SEQ ID NO: 16, and an LCDR1, an LCDR2, and an LCDR3 of the sequence set forth in SEQ ID NO: 17, or sequences having at least 70% identity or at most 3 mutations compared thereto; or
(2) an HCDR1, an HCDR2, and an HCDR3 of the sequence set forth in SEQ ID NO: 18, and an LCDR1, an LCDR2, and an LCDR3 of the sequence set forth in SEQ ID NO: 19, or sequences having at least 70% identity or at most 3 mutations compared thereto; or
(3) an HCDR1, an HCDR2, and an HCDR3 of the sequence set forth in SEQ ID NO: 20, or sequences having at least 70% identity or at most 3 mutations compared thereto; or
(4) an HCDR1, an HCDR2, and an HCDR3 of the sequence set forth in SEQ ID NO: 71, and an LCDR1, an LCDR2, and an LCDR3 of the sequence set forth in SEQ ID NO: 72, or sequences having at least 70% identity or at most 3 mutations compared thereto; or
(5) an HCDR1, an HCDR2, and an HCDR3 of the sequence set forth in SEQ ID NO: 73, and an LCDR1, an LCDR2, and an LCDR3 of the sequence set forth in SEQ ID NO: 74, or sequences having at least 70% identity or at most 3 mutations compared thereto.

Preferably, the HCDR1-3 and/or the LCDR1-3 are determined according to the Kabat, Chothia, or IMGT scheme.

In some embodiments, the antigen-binding domain specifically binding to IL23 in the molecule comprises a heavy chain variable region and/or a light chain variable region of the antibody.

In some specific embodiments, the heavy chain variable region has the sequence set forth in any one of SEQ ID NO: 16, 18, 20, 71, or 73, or a sequence having at least 70% identity or at most 15 mutations compared thereto; and/or the light chain variable region has the sequence set forth in any one of SEQ ID NO: 17, 19, 72, or 74, or a sequence having at least 70% identity or at most 15 mutations compared thereto.

In some specific embodiments, the Fc region has the amino acid sequence set forth in any one of SEQ ID NOs: 21-27, or a sequence having at least 70% identity or at most 15 mutations compared thereto.

In some specific embodiments, the CH1 region has the amino acid sequence set forth in SEQ ID NO: 28, or a sequence having at least 70% identity or at most 15 mutations compared thereto.

In some specific embodiments, the CL region has an amino acid sequence set forth in SEQ ID NO: 29 or 30, or a sequence having at least 70% identity or at most 15 mutations compared thereto.

In some specific embodiments, the Fc region of the molecule comprises an amino acid mutation, wherein preferably, the amino acid mutation comprises an L234A, L235A, and/or G237A mutation; preferably, the amino acid mutation comprises an M252Y, S254T, and/or T256E mutation; more preferably, the amino acid mutation that alters effector functions comprises: (a) an L234A and/or L235A mutation of IgG1 Fc; (b) an L234A, L235A, and/or G237A mutation of IgG1 Fc; and/or (c) an L234A, L235A, M252Y, S254T, and/or T256E mutation of IgG1 Fc.

In some specific embodiments, the Fc region of the molecule has an amino acid mutation that promotes heterogeneous dimerization, wherein preferably, the amino acid mutation that promotes heterogeneous dimerization comprises a knob-into-hole (KIH) mutation; more preferably, the knob-into-hole mutation comprises: (a) any one of the Fc regions comprising a T366W mutation; and (b) the other Fc region comprising a T366S, L368A, and/or Y407V mutation.

In some embodiments, the molecule may be bispecific, trispecific, or tetraspecific.

In some embodiments, the molecule may be divalent, trivalent, tetravalent, pentavalent, or hexavalent.

In some specific embodiments, the at least 70% identity is at least 70%, 75%, 80%, 85%, 90%, 91%, 92%, 93%, 94%, 95%, 96%, 97%, 98%, 99%, or 100% identity; the at most 3 mutations are preferably at most 3, 2, 1, or 0 mutations; the at most 15 mutations are at most 15, 14, 13, 12, 11, 10, 9, 8, 7, 6, 5, 4, 3, 2, 1, or 0 mutations.

In some embodiments, the mutation is an insertion, a deletion, or a substitution, wherein the substitution is preferably a conservative amino acid substitution; the mutation is preferably a back mutation or a hotspot mutation.

In some specific embodiments, the bispecific molecule described in the present disclosure comprises:
(1) a chimeric antibody or a fragment thereof; and/or
(2) a humanized antibody or a fragment thereof; and/or
(3) a fully human antibody or a fragment thereof.

In a second aspect, the present disclosure provides an isolated nucleic acid, which encodes the molecule described above.

In a third aspect, the present disclosure provides a recombinant vector, which comprises the isolated nucleic acid described above.

In a fourth aspect, the present disclosure provides a host cell, which comprises the isolated nucleic acid described above or the recombinant vector described above, wherein preferably, the host cell is a eukaryotic cell or a prokaryotic cell; more preferably, the host cell is derived from a mammalian cell, a yeast cell, an insect cell, E. coli, and/or Bacillus subtilis; more preferably, the host cell is an Expi293 cell.

In a fifth aspect, the present disclosure provides a method for preparing the molecule described above, which comprises culturing the host cell described above under suitable conditions and isolating the molecule.

In a sixth aspect, the present disclosure provides a pharmaceutical composition, which comprises the molecule described above, the isolated nucleic acid described above, the recombinant vector described above, the host cell described above, or a product prepared by the method described above, wherein preferably, the pharmaceutical composition further comprises one or more pharmaceutically acceptable carriers, diluents, buffers, or excipients; preferably, the pharmaceutical composition further comprises an additional therapeutic agent.

In a seventh aspect, the present disclosure provides use of the molecule described above, the isolated nucleic acid described above, the host cell described above, a product prepared by the method described above, or the pharmaceutical composition described above in preparing a medicament for treating an immune-mediated inflammatory disease (IMID).

Preferably, the immune-mediated inflammatory disease (IMID) includes, but is not limited to, one or more of inflammatory bowel disease (e.g., Crohn's disease and ulcerative colitis), psoriasis, rheumatoid arthritis, psoriatic arthritis, arteriosclerosis, uveitis, and the like.

In an eighth aspect, the present disclosure provides a method for treating and/or preventing an immune-mediated inflammatory disease (IMID), which comprises administering to a subject a therapeutically effective amount of the antigen-binding molecule described above, the isolated nucleic acid described above, the host cell described above, a product prepared by the method described above, or the pharmaceutical composition described above.

Preferably, the immune-mediated inflammatory disease (IMID) includes, but is not limited to, one or more of inflammatory bowel disease (e.g., Crohn's disease and ulcerative colitis), psoriasis, rheumatoid arthritis, psoriatic arthritis, arteriosclerosis, uveitis, and the like.

In a ninth aspect, the present disclosure provides the molecule described above, the isolated nucleic acid described above, the host cell described above, a product prepared by the method described above, or the pharmaceutical composition described above for use in treating and/or preventing an immune-mediated inflammatory disease (IMID).

Preferably, the immune-mediated inflammatory disease (IMID) includes, but is not limited to, one or more of inflammatory bowel disease (e.g., Crohn's disease and ulcerative colitis), psoriasis, rheumatoid arthritis, psoriatic arthritis, arteriosclerosis, uveitis, and the like.

### Numbered Paragraphs

The following numbered paragraphs provide further statements of features and combinations of features which are contemplated in connection with the present invention:

1. A molecule specifically binding to TL1A and IL23, comprising an antibody or an antigen-binding fragment specifically binding to TL1A and IL23.

2. The molecule according to paragraph 1, comprising a first antigen-binding domain ABD1 and a second antigen-binding domain ABD2, wherein the ABD1 and the ABD2 specifically bind to TL1A or IL23.

3. The molecule according to paragraph 1 or 2, wherein the ABD1 and the ABD2 are each independently selected from an antibody, an antibody fragment, a F(ab′) 2, a Fab′, a Fab, an Fv, an scFv, a nanobody, and VHH.

4. The molecule according to any one of paragraphs 1-3, wherein the first antigen-binding domain ABD1 comprises a heavy chain variable region VH1 and/or a light chain variable region VL1, and the second antigen-binding domain ABD2 comprises a heavy chain variable region VH2 and/or a light chain variable region VL2.

5. The molecule according to paragraph 4, comprising the following structure:
(1) an IgG antibody and an scFv structure, wherein the scFv structure can be located at the N-terminus or C-terminus of a heavy chain or a light chain of the IgG antibody; or
(2) an IgG antibody and a VHH structure, wherein the VHH structure can be located at the N-terminus or C-terminus of a heavy chain or a light chain of the IgG antibody; or
(3) the molecule being a “Crossmab” type antibody; or
(4) a Fab, an Fc region, and a VHH structure, wherein the VHH structure can be located on either of the two chains of the Fc region, wherein
preferably, the molecule can be bispecific, trispecific, or tetraspecific;
preferably, the molecule can be divalent, trivalent, tetravalent, pentavalent, or hexavalent.

6. The molecule according to paragraph 5, wherein (1) a first heavy chain and a second heavy chain of the molecule are identical and have a structure as shown below:

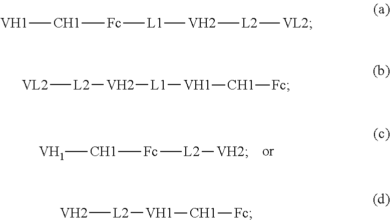

and/or
(2) a first light chain and a second light chain of the molecule are identical and have the following structure: VL1-CL,
wherein the VH1/VH2 and/or the VL1/VL2 are optionally heavy chain variable regions and/or light chain variable regions specifically binding to TL1A or IL23, and the Fc is an Fc region of any one of the antibodies; the two light chains comprise VL1-CL; the L1 and the L2 are identical or different linkers;
preferably, the L1 is (G4S) 4 (SEQ ID NO: 31), and the L2 is (G4S) 3 (SEQ ID NO: 32).

7. The molecule according to paragraph 5, comprising a first heavy chain and a second heavy chain, and a first light chain and a second light chain, wherein the first heavy chain comprises VH1-CH1-Fc, the second heavy chain comprises VH2-CL-Fc, the first light chain comprises VL1-CL, the second light chain comprises VL2-CH1, and the VH1/VH2 and/or the VL1/VL2 are optionally heavy chain variable regions and/or light chain variable regions specifically binding to TL1A or IL23.

8. The molecule according to paragraph 5, comprising three polypeptide chains: a first heavy chain, a second heavy chain, and a light chain, wherein (1) the first heavy chain and the second heavy chain of the molecule have a structure as shown below:
(a) the first heavy chain VH1-CH1-Fc and the second heavy chain VH2-Fc;
(b) the first heavy chain VH1-CH1-Fc-VH2 and the second heavy chain Fc; or
(c) the first heavy chain VH1-CH1-Fc and the second heavy chain Fc-VH2; and/or
(2) the light chain of the molecule has the following structure: VL1-CL,
wherein the VH1/VH2 and/or the VL1/VL2 are optionally heavy chain variable regions and/or light chain variable regions specifically binding to TL1A or IL23, and the Fc is an Fc region of any one of the antibodies.

9. The molecule according to any one of paragraphs 1-8, wherein the antigen-binding domain specifically binding to TL1A in the molecule comprises a heavy chain variable region and/or a light chain variable region of the antibody as follows:
(a) the heavy chain variable region comprising an HCDR1, an HCDR2, and an HCDR3, wherein the HCDR1, HCDR2, and HCDR3 have the HCDR1, the HCDR2, and the HCDR3 of a sequence set forth in any one of SEQ ID NO: 9, 11, 13, 15, 69, or 70; and/or
(b) the light chain variable region comprising an LCDR1, an LCDR2, and an LCDR3, wherein the LCDR1, LCDR2, and LCDR3 have the LCDR1, the LCDR2, and the LCDR3 of a sequence set forth in any one of SEQ ID NO: 10, 12, or 14.

10. The molecule according to any one of paragraphs 1-9, wherein the antigen-binding domain specifically binding to TL1A in the molecule comprises a heavy chain variable region and/or a light chain variable region of the antibody, wherein
(a) the heavy chain variable region comprises an HCDR1, an HCDR2, and an HCDR3, wherein the HCDR1, the HCDR2, and the HCDR3 have sequences set forth in SEQ ID NOs: 36-38, SEQ ID NOs: 42-44, SEQ ID NOs: 48-50, or SEQ ID NOs: 51-53, and SEQ ID NOs: 75-77 or SEQ ID NOs: 78-80; and/or
(b) the light chain variable region comprises an LCDR1, an LCDR2, and an LCDR3, wherein the LCDR1, the LCDR2, and the LCDR3 have sequences set forth in SEQ ID NOs: 33-35, SEQ ID NOS: 39-41, or SEQ ID NOs: 45-47.

11. The molecule according to any one of paragraphs 1-10, wherein the antigen-binding domain specifically binding to TL1A in the molecule comprises:
(1) an HCDR1, an HCDR2, and an HCDR3 of the sequence set forth in SEQ ID NO: 9, and an LCDR1, an LCDR2, and an LCDR3 of the sequence set forth in SEQ ID NO: 10; or
(2) an HCDR1, an HCDR2, and an HCDR3 of the sequence set forth in SEQ ID NO: 11, and an LCDR1, an LCDR2, and an LCDR3 of the sequence set forth in SEQ ID NO: 12; or
(3) an HCDR1, an HCDR2, and an HCDR3 of the sequence set forth in SEQ ID NO: 13, and an LCDR1, an LCDR2, and an LCDR3 of the sequence set forth in SEQ ID NO: 14; or
(4) an HCDR1, an HCDR2, and an HCDR3 of the sequence set forth in SEQ ID NO: 15; or
(5) an HCDR1, an HCDR2, and an HCDR3 of the sequence set forth in SEQ ID NO: 69; or
(6) an HCDR1, an HCDR2, and an HCDR3 of the sequence set forth in SEQ ID NO: 70.

12. The molecule according to any one of paragraphs 1-11, wherein the antigen-binding domain specifically binding to TL1A in the molecule comprises a heavy chain variable region and/or a light chain variable region of the antibody as follows:
the heavy chain variable region has the sequence set forth in any one of SEQ ID NO: 9, 11, 13, 15, 69, or 70, or a sequence having at least 70% identity or at most 15 mutations compared thereto; and/or the light chain variable region has the sequence set forth in any one of SEQ ID NO: 10, 12, or 14, or a sequence having at least 70% identity or at most 15 mutations compared thereto.

13. The molecule according to any one of paragraphs 1-12, wherein the antigen-binding domain specifically binding to IL23 in the molecule comprises a heavy chain variable region and/or a light chain variable region of the antibody as follows:
(a) the heavy chain variable region comprising an HCDR1, an HCDR2, and an HCDR3, wherein the HCDR1, HCDR2, and HCDR3 have the HCDR1, the HCDR2, and the HCDR3 of a sequence set forth in any one of SEQ ID NO: 16, 18, 20, 71, or 73, or a sequence having at least 70% identity or at most 3 mutations compared thereto; and/or
(b) the light chain variable region comprising an LCDR1, an LCDR2, and an LCDR3, wherein the LCDR1, LCDR2, and LCDR3 have the LCDR1, the LCDR2, and the LCDR3 of a sequence set forth in any one of SEQ ID NO: 17, 19, 72, or 74, or a sequence having at least 70% identity or at most 3 mutations compared thereto.

14. The molecule according to any one of paragraphs 1-13, wherein the antigen-binding domain specifically binding to IL23 in the molecule comprises a heavy chain variable region and/or a light chain variable region of the antibody as follows:
(a) the heavy chain variable region comprises an HCDR1, an HCDR2, and an HCDR3, wherein the HCDR1, the HCDR2, and the HCDR3 have sequences set forth in SEQ ID NOs: 54-56, SEQ ID NOS: 60-62, SEQ ID NOs: 66-68, SEQ ID NOs: 54, 81, and 56, or SEQ ID NOs: 54, 84, and 56; and/or
(b) the light chain variable region comprises an LCDR1, an LCDR2, and an LCDR3, wherein the LCDR1, the LCDR2, and the LCDR3 have sequences set forth in SEQ ID NOs: 57-59, SEQ ID NOS: 63-65, SEQ ID NOs: 82, 83, and 59, or SEQ ID NOs: 85, 58, and 59.

15. The molecule according to any one of paragraphs 1-14, wherein the antigen-binding domain specifically binding to IL23 in the molecule comprises:
(1) an HCDR1, an HCDR2, and an HCDR3 of the sequence set forth in SEQ ID NO: 16, and an LCDR1, an LCDR2, and an LCDR3 of the sequence set forth in SEQ ID NO: 17; or
(2) an HCDR1, an HCDR2, and an HCDR3 of the sequence set forth in SEQ ID NO: 18, and an LCDR1, an LCDR2, and an LCDR3 of the sequence set forth in SEQ ID NO: 19; or
(3) an HCDR1, an HCDR2, and an HCDR3 of the sequence set forth in SEQ ID NO: 20; or
(4) an HCDR1, an HCDR2, and an HCDR3 of the sequence set forth in SEQ ID NO: 71, and an LCDR1, an LCDR2, and an LCDR3 of the sequence set forth in SEQ ID NO: 72; or
(5) an HCDR1, an HCDR2, and an HCDR3 of the sequence set forth in SEQ ID NO: 73, and an LCDR1, an LCDR2, and an LCDR3 of the sequence set forth in SEQ ID NO: 74.

16. The molecule according to any one of paragraphs 1-14, wherein the antigen-binding domain specifically binding to IL23 in the molecule comprises a heavy chain variable region and/or a light chain variable region of the antibody as follows:
the heavy chain variable region has the sequence set forth in any one of SEQ ID NO: 16, 18, 20, 71, or 73, or a sequence having at least 70% identity or at most 15 mutations compared thereto; and/or the light chain variable region has the sequence set forth in any one of SEQ ID NO: 17, 19, 72, or 74, or a sequence having at least 70% identity or at most 15 mutations compared thereto.

17. The molecule according to any one of paragraphs 9-11 and 13-15, wherein the HCDR1-3 and/or the LCDR1-3 are determined according to the Kabat, Chothia, or IMGT scheme.

18. The molecule according to any one of paragraphs 1-17, wherein the Fc region has the amino acid sequence set forth in any one of SEQ ID NOs: 21-27, or a sequence having at least 70% identity or at most 15 mutations compared thereto; and/or the CH1 region has the amino acid sequence set forth in SEQ ID NO: 28, or a sequence having at least 70% identity or at most 15 mutations compared thereto; and/or the CL region has the amino acid sequence set forth in SEQ ID NO: 29 or 30, or a sequence having at least 70% identity or at most 15 mutations compared thereto.

19. The molecule according to paragraph 18, wherein the Fc region of the molecule comprises the following amino acid mutation:
(1) an amino acid mutation that alters effector functions, wherein preferably, the amino acid mutation comprises an L234A, L235A, and/or G237A mutation; preferably, the amino acid mutation comprises an M252Y, S254T, and/or T256E mutation; more preferably, the amino acid mutation that alters effector functions comprises: (a) an L234A and/or L235A mutation of IgG1 Fc; (b) an L234A, L235A, and/or G237A mutation of IgG1 Fc; and/or (c) an L234A, L235A, M252Y, S254T, and/or T256E mutation of IgG1 Fc; and/or
(2) an amino acid mutation that promotes heterogeneous dimerization, wherein preferably, the amino acid mutation that promotes heterogeneous dimerization comprises a knob-into-hole (KIH) mutation; more preferably, the knob-into-hole mutation comprises: (a) any one of the Fc regions comprising a T366W mutation; and (b) the other Fc region comprising a T366S, L368A, and/or Y407V mutation.

20. The molecule according to any one of paragraphs 1-19, wherein the at least 70% identity is at least 70%, 75%, 80%, 85%, 90%, 91%, 92%, 93%, 94%, 95%, 96%, 97%, 98%, 99%, or 100% identity, the at most 3 mutations are at most 3, 2, 1, or 0 mutations, and the at most 15 mutations are at most 15, 14, 13, 12, 11, 10, 9, 8, 7, 6, 5, 4, 3, 2, 1, or 0 mutations; preferably, the mutation is selected from substitution, deletion, and insertion mutations; preferably, the mutation is a back mutation or a hotspot mutation; preferably, the substitution is a conservative amino acid substitution.

21. The molecule according to any one of paragraphs 1-20, comprising: (1) a chimeric antibody or a fragment thereof; (2) a humanized antibody or a fragment thereof; and/or (3) a fully human antibody or a fragment thereof.

22. An isolated nucleic acid, encoding the molecule according to any one of paragraphs 1-21.

23. A vector, comprising the nucleic acid according to paragraph 22.

24. A cell, comprising the vector according to paragraph 23.

25. A method for preparing the molecule according to any one of paragraphs 1-21, comprising: (1) culturing the cell according to paragraph 24 and/or (2) isolating a molecule expressed by the cell.

26. A pharmaceutical composition, comprising the molecule according to any one of paragraphs 1-21, or the nucleic acid according to paragraph 22, or the vector according to paragraph 23, or the cell according to paragraph 24, or a product prepared by the method according to paragraph 25, wherein preferably, the composition further comprises a pharmaceutically acceptable carrier, diluent, buffer, or excipient; preferably, the pharmaceutical composition further comprises an additional therapeutic agent.

27. Use of the molecule according to any one of paragraphs 1-21, the nucleic acid according to paragraph 22, the vector according to paragraph 23, the cell according to paragraph 24, or a product prepared by the method according to paragraph 25 in preparing a medicament for treating an immune-mediated inflammatory disease (IMID), wherein preferably, the immune-mediated inflammatory disease (IMID) is an autoimmune disease and/or an inflammatory disease; more preferably, the immune-mediated inflammatory disease (IMID) can be selected from one or more of inflammatory bowel disease, psoriasis, rheumatoid arthritis, psoriatic arthritis, arteriosclerosis, uveitis, and the like.

28. A method for treating an immune-mediated inflammatory disease (IMID), comprising administering to a subject an effective amount of a medicament comprising the molecule according to any one of paragraphs 1-21, the nucleic acid according to paragraph 22, the vector according to paragraph 23, the cell according to paragraph 24, or a product prepared by the method according to paragraph 25, wherein preferably, the immune-mediated inflammatory disease (IMID) is an autoimmune disease and/or an inflammatory disease; more preferably, the immune-mediated inflammatory disease (IMID) can be selected from one or more of inflammatory bowel disease, psoriasis, rheumatoid arthritis, psoriatic arthritis, arteriosclerosis, uveitis, and the like.

29. The molecule according to any one of paragraphs 1-21, the nucleic acid according to paragraph 22, the vector according to paragraph 23, the cell according to paragraph 24, or a product prepared by the method according to paragraph 25 for use in treating an immune-mediated inflammatory disease (IMID), wherein preferably, the immune-mediated inflammatory disease (IMID) is an autoimmune disease and/or an inflammatory disease; more preferably, the immune-mediated inflammatory disease (IMID) can be selected from one or more of inflammatory bowel disease, psoriasis, rheumatoid arthritis, psoriatic arthritis, arteriosclerosis, uveitis, and the like.

### Terminology and Definitions

Unless otherwise defined herein, scientific and technical terms used in correlation with the present disclosure shall have the meanings that are commonly understood by those skilled in the art.

Furthermore, unless otherwise stated herein, terms used in the singular form herein shall include the plural form, and vice versa. More specifically, as used in this specification and the appended claims, unless otherwise clearly indicated, the singular forms “a”, “an”, and “the” include referents in the plural form.

The terms “include”, “comprise”, and “have” herein are used interchangeably and are intended to indicate the inclusion of a solution, implying that there may be elements other than those listed in the solution. Meanwhile, it should be understood that the descriptions “include”, “comprise”, and “have” used herein also provide the solution of “consist of”. Illustratively, “a composition, comprising A and B” should be interpreted as the following technical solution: a composition consisting of A and B, and a composition containing other components in addition to A and B, all of which fall within the scope of the “composition” described above.

The term “and/or” used herein includes the meanings of “and”, “or”, and “all or any other combination of elements linked by the term”.

The term “TLIA”, also known as “TNF ligand-associated molecule 1 and vascular endothelial cell growth inhibitor (VEGI)” or “tumor necrosis factor superfamily member 15 (TNFSF15)” herein, belongs to the tumor necrosis factor family, and is a ligand of DR3 and decoy receptor TR6/DcR3. “TL1A” herein includes full-length TL1A proteins or mutants thereof (e.g., point mutation, insertion mutation, or deletion mutation), splice variants, orthologs, and TL1A fragments. “TL1A” herein may be derived from humans, primates (e.g., cynomolgus monkeys and rhesus monkeys), and rodents (e.g., mice and rats). Illustratively, an amino acid sequence of human TL1A can be found in Uniprot No.: O95159, an amino acid sequence of cynomolgus monkey TL1A can be found in Uniprot No.: G7PRK8, and an amino acid sequence of mouse TL1A can be found in Uniprot No.: Q5UBV8.

The term “IL-23or “IL23” herein, also known as “interleukin 23” or “interleukin-23”, includes a heterodimeric protein comprising a 19 KD subunit (p19) and a 40 KD subunit (p40), both of which are linked together by a disulfide bridge. “IL-12p40” is identical to “IL-23p40” and is also simply referred to as “p40” and “p40 subunit” or “IL12B”, including the 40 kD subunit (p40) of the human cytokine IL-12 and the 40 kD subunit of the human cytokine IL-23; “IL-23p19”, also simply referred to as “p19” or “IL23A”, includes the 19 kD subunit of the human cytokine IL-23. IL23A and IL12B may be derived from humans, primates (e.g., cynomolgus monkeys and rhesus monkeys), and rodents (e.g., mice and rats). Illustratively, an amino acid sequence of human IL23A can be found in Uniprot No.: Q9NPF7, an amino acid sequence of cynomolgus monkey IL23A can be found in NCBI No.: NP_001274588.1, and an amino acid sequence of mouse IL23A can be found in Uniprot No.: Q9EQ14. An amino acid sequence of human IL12B can be found in Uniprot No.: P29460, an amino acid sequence of cynomolgus monkey IL12B can be found in NCBI No.: NP_001274204.1, and an amino acid sequence of mouse IL12B can be found in Uniprot No.: P43432.

The IL-12R complex consists of two subunits: IL-12 receptor β1 (IL-12RB1) and IL-12 receptor β2 (IL-12RB2), both of which are required for high-affinity binding and signaling of IL-12. The IL-23R complex consists of two subunits: IL-12RB1 and the IL-23 receptor (IL-23R). The engagement of IL-23 with IL-23R leads to autophosphorylation of JAK2 and phosphorylation of IL-23R. This results in the localization and phosphorylation of STAT3, as well as STAT1, STAT4, and STAT5.

The terms “specific binding”, “immunobinding”, and “immunobinding property” herein refer to the type of non-covalent interaction formed between an immunoglobulin molecule and an antigen to which the immunoglobulin has specificity. An antigen-binding molecule (e.g., a body) specifically binds to an antigen and substantially identical antigens, generally with high affinity, but does not bind to unrelated antigens with high affinity. The strength or affinity of an immunobinding interaction is generally reflected by an equilibrium dissociation constant (KD), where relatively low KD indicates relatively high affinity. The immunobinding properties of a selected polypeptide can be quantitatively determined using methods well known in the art. In the case of antibodies, high affinity generally means having KD of about 10⁻⁸ M or less, about 1×10⁻⁹ M or less, about 1×10⁻¹⁰ M or less, 1×10⁻¹¹ M or less, or 1×10⁻¹² M or less. The KD is calculated as follows: KD=Kd/Ka, where Kd represents the dissociation rate, and Ka represents the association rate. The equilibrium dissociation constant KD can be measured by methods well known in the art, such as surface plasmon resonance (e.g., Biacore) or equilibrium dialysis. Illustratively, KD can be obtained by the method as described in Example 8 herein.

The term “antigen-binding molecule” herein is used in its broadest sense and refers to a molecule that specifically binds to an antigen. Illustratively, the antigen-binding molecule includes, but is not limited to, an antibody or an antibody mimetic. “Antibody mimetic” refers to an organic compound or a binding domain that is capable of specifically binding to an antigen, but is not structurally related to an antibody. Illustratively, the antibody mimetic includes, but is not limited to, affibody, affitin, affilin, a designed ankyrin repeat protein (DARPin), a nucleic acid aptamer, or a Kunitz domain peptide.

The term “antibody” herein is used in its broadest sense and refers to a polypeptide or a combination of polypeptides that comprises a sufficient sequence from an immunoglobulin heavy chain variable region and/or a sufficient sequence from an immunoglobulin light chain variable region to be capable of specifically binding to an antigen. The “antibody” herein encompasses various forms and various structures as long as they exhibit the desired antigen-binding activity. The terms “full-length antibody”, “complete antibody”, and “intact antibody” are used interchangeably herein and refer to an antibody having a structure substantially similar to that of a natural antibody. “Antibody” herein includes, but is not limited to, monoclonal antibodies, polyclonal antibodies, monospecific antibodies, multispecific antibodies (e.g., bispecific antibodies), monovalent antibodies, multivalent antibodies, intact antibodies, fragments of an intact antibody, naked antibodies, conjugated antibodies, chimeric antibodies, humanized antibodies, or fully human antibodies.

The terms “antibody fragment” and “antigen-binding fragment” are used interchangeably herein to refer to a portion of an antibody, which does not have the entire structure of an intact antibody, but only comprises a portion of the intact antibody or a variant of the portion that has the ability to bind to the same antigen recognized by the intact antibody. “Antibody fragment” or “antigen-binding fragment” also includes any synthetic or genetically engineered protein that acts like an antibody by binding to a specific antigen to form a complex. For example, antibody fragments include isolated fragments consisting of the light chain variable region, “Fv” fragments consisting of the heavy and light chain variable regions, recombinant single chain polypeptide molecules (scFv) in which the light and heavy chain variable regions are linked via a peptide linker, and minimal recognition units consisting of amino acid residues that mimic a hypervariable region. Illustratively, “antigen-binding fragment” or “antibody fragment” herein includes, but is not limited to, a Fab, a F(ab′)2, a Fab′, a Fab′-SH, an Fd, an Fv, an scFv, a diabody, and a single domain antibody.

“Antibody” herein further includes alternative protein scaffolds or artificial scaffolds having grafted complementarity determining regions (CDRs) or CDR derivatives. Such scaffolds include antibody-derived scaffolds comprising mutations introduced to, for example, stabilize the three-dimensional structure of the antibody, and fully synthetic scaffolds comprising, for example, biocompatible polymers. See, e.g., Korndorfer et al., 2003, Proteins: Structure, Function, and Bioinformatics, 53 (1): 121-129 (2003); and Roque et al., Biotechnol. Prog. 20:639-654 (2004). Such scaffolds may also include non-antibody-derived scaffolds, such as scaffold proteins known in the art to be useful for grafting CDRs, including but not limited to tenascin, fibronectin, peptide aptamers, and the like.

“Antibody” herein includes a typical “tetrabody”, which belongs to an immunoglobulin consisting of two heavy chains (HCs) and two light chains (LCs). The heavy chain refers to a polypeptide chain consisting of, from the N-terminus to the C-terminus, a heavy chain variable region (VH), a heavy chain constant region CH1 domain, a hinge region (HR), a heavy chain constant region CH2 domain, and a heavy chain constant region CH3 domain; moreover, when the full-length antibody is of IgE isoform, the heavy chain optionally further comprises a heavy chain constant region CH4 domain. The light chain is a polypeptide chain consisting of, from the N-terminus to the C-terminus, a light chain variable region (VL) and a light chain constant region (CL). The heavy chains are linked to each other and to the light chains through disulfide bonds to form a Y-shaped structure. The heavy chain constant regions of immunoglobulins differ in their amino acid composition and arrangement, and thus in their antigenicity. Accordingly, “immunoglobulin” herein can be divided into five classes, or referred to as isoforms of immunoglobulins, i.e., IgM, IgD, IgG, IgA, and IgE, with their corresponding heavy chains being μ, δ, γ, α, and ε chains, respectively. The Ig of the same class may also be divided into different subclasses according to the differences in the amino acid composition of the hinge regions and the number and location of disulfide bonds in the heavy chains. For example, IgG may be divided into IgG1, IgG2, IgG3, and IgG4, and IgA may be divided into IgA1 and IgA2. Light chains are divided into κ chains or λ chains according to differences in the constant regions. Each of the five classes of Ig may have a κ chain or λ chain.

“Antibody” herein also includes antibodies that do not comprise a light chain, e.g., heavy-chain antibodies (HCAbs) produced by Camelus dromedarius, Camelus bactrianus, Lama glama, Lama guanicoe, and Vicugna pacos, as well as Ig new antigen receptors (IgNARs) found in Chondrichthyes, e.g., shark.

The antibody herein also includes “CrossMab” type antibodies, which are multispecific antibodies having domain substitutions/exchanges in one binding arm (CrossMabVH-VL or CrossMabCH-CL). These are detailed in WO2009/080252 and Schaefer, W. et al., PNAS, 108 (2011) 11187-1191. This technique can effectively solve the problem of light chain mispairing of bispecific antibodies. CrossMab is an IgG-like bispecific antibody. It retains the general structure and size of traditional IgG and thus has biophysical properties similar to those of IgG. In the CrossMAb, the KIH (knob-into-hole) technology is used to allow heavy chain heterodimerization. Light chain mispairing can be overcome by the crossover of antibody domains, the crossover of entire Fab domains, and the crossover of variable domains or constant domains only. The crossover can maintain the antigen-binding ability of the parent antibody. In other words, the “CrossMab” strategy can well circumvent the “light chain mispairing” problem by constructing correct light chain associations through the exchange of the CL (light chain constant region) domain of the light chain with the corresponding CH1 domain of the heavy chain. Domain exchanges can also occur between VH-CH1 and VL-CL and between VH and VL. Additionally, based on the need for high yield and reduced byproducts, charges can be introduced into the non-crossover CH1-CL.

“Antibody” herein may be derived from any animal, including, but not limited to, human and non-human animals, wherein the non-human animals may be selected from primates, mammals, rodents, and vertebrates, such as Camelidae species, Lama glama, Lama guanicoe, Vicugna pacos, sheep, rabbits, mice, rats, or Chondrichthyes (e.g., shark).

The term “monoclonal antibody” herein refers to an antibody obtained from a population of substantially homogeneous antibodies, that is, the individual antibodies constituting the population are identical and/or bind to the same epitope, except for possible variants (e.g., containing naturally occurring mutations or arising during the production of the formulation, such variants typically being present in minor amounts). In contrast to polyclonal antibody formulations, which generally comprise different antibodies directed against different determinants (epitopes), each monoclonal antibody in a monoclonal antibody formulation is directed against a single determinant on an antigen. The modifier “monoclonal” herein is not to be construed as requiring the antibody or the antigen-binding molecule to be produced by any particular method. For example, monoclonal antibodies can be prepared by a variety of techniques, including (but not limited to) a hybridoma technique, a recombinant DNA method, a phage library display technique, methods that utilize transgenic animals containing all or part of human immunoglobulin loci, and other methods known in the art.

The term “natural antibody” herein refers to an antibody that is produced and paired by the immune system of a multicellular organism. The term “engineered antibody” herein refers to a non-natural antibody obtained by genetic engineering, antibody engineering, and the like. Illustratively, “engineered antibody” includes chimeric antibodies, humanized antibodies, antibody fragments (e.g., scFv and sdAb), bispecific antibodies, and the like.

The term “monospecific” means having one or more binding sites, each of which binds to the same epitope of the same antigen.

The term “multispecific antibody” means having at least two antigen-binding sites, each of which binds to a different epitope of the same antigen or a different epitope of a different antigen. Thus, the terms such as “bispecific”, “trispecific”, and “tetraspecific” refer to the number of different epitopes to which an antibody/antigen-binding molecule can bind.

The term “valent” refers to the presence of a specified number of binding sites in an antibody/antigen-binding molecule. Thus, the terms “monovalent”, “divalent”, “trivalent”, “tetravalent”, “pentavalent”, and “hexavalent” refer to the presence of one binding site, two binding sites, three binding sites, four binding sites, five binding sites, and six binding sites, respectively, in an antibody/antigen-binding molecule.

An intact antibody is digested by papain to produce two identical antigen-binding fragments, called “Fab” fragments, each of which contains a heavy chain variable domain and a light chain variable domain, as well as a light chain constant domain and a first heavy chain constant domain (CH1). Thus, the term “Fab fragment” herein refers to an antibody fragment comprising a light chain fragment comprising the VL domain and the constant domain (CL) of a light chain, and the VH domain and the first constant domain (CH1) of a heavy chain. A Fab′ fragment differs from the Fab fragment by the addition of a few residues (including one or more cysteines from an antibody hinge region) at the carboxyl terminus of the heavy chain CH1 domain. Fab′-SH is a Fab′ fragment in which the cysteine residue in the constant domain carries a free thiol group. Pepsin treatment produces a F(ab′)2 fragment having two antigen-binding sites (two Fab fragments) and a portion of the Fc region. The term “Fd” herein refers to an antibody consisting of VH and CH1 domains. The term “Fv” herein refers to an antibody fragment consisting of VL and VH domains of a single arm. An Fv fragment is generally considered to be the smallest antibody fragment capable of forming an intact antigen-binding site. An Fv fragment has the same binding properties and similar three-dimensional binding properties as Fab. The VH and VL chains of the Fv fragment are bound together by a non-covalent interaction. It is generally believed that the six CDRs provide antigen-binding specificity to the antibody. However, even one variable region (e.g., an Fd fragment, which contains only three CDRs specific to an antigen) is also capable of recognizing and binding to an antigen, although its affinity may be lower than that of an intact binding site.

The term “scFv (single-chain variable fragment)” herein refers to a single polypeptide chain comprising VL and VH domains, wherein the VL and VH are linked via a linker. Such scFv molecules may have a general structure: NH2-VL-linker-VH—COOH or NH2-VH-linker-VL-COOH. An appropriate linker in the prior art consists of GGGGS amino acid sequence repeats or a variant thereof. For example, a linker having the amino acid sequence (GGGGS) 4 may be used, but a variant thereof may also be used. Other linkers that may be used in the present disclosure are described in Alfthan et al. (1995), Protein Eng. 8: 725-731; Choi et al. (2001), Eur. J. Immunol. 31:94-106; Hu et al. (1996), Cancer Res. 56: 3055-3061; Kipriyanov et al. (1999), J. Mol. Biol. 293: 41-56; and Roovers et al. (2001), Cancer Immunol. In some cases, there may also be disulfide bonds between the VH and VL of the scFv, forming a disulfide-linked Fv (dsFv).

The term “diabody” herein refers to an antibody having VH and VL domains expressed on a single polypeptide chain, but using a linker that is too short to allow pairing between the two domains on the same chain, thereby forcing the domains to pair with complementary domains of another chain and producing two antigen-binding sites.

The terms “single domain antibody” (sdAb), “VHH”, and “nanobody” herein have the same meaning and are used interchangeably, and refer to a single domain antibody consisting of only one heavy chain variable region constructed by cloning a variable region of an antibody heavy chain, which is the smallest antigen-binding fragment with complete function. Generally, a single domain antibody consisting of only one heavy chain variable region is constructed by acquiring an antibody naturally lacking a light chain and a heavy chain constant region 1 (CH1) and then cloning the variable region of the antibody heavy chain. The single domain antibody may be derived from a Camelidae heavy-chain antibody or Chondrichthyes IgNAR.

The term “chimeric antibody” herein refers to an antibody in which a portion of the light or/and heavy chain is derived from one antibody (which may be derived from a particular species or belong to a particular antibody class or subclass) and another portion of the light or/and heavy chain is derived from another antibody (which may be derived from the same or a different species or belong to the same or a different antibody class or subclass), but which nevertheless retains binding activity to a target antigen. For example, the term “chimeric antibody” may include an antibody (e.g., a human-murine chimeric antibody) in which the heavy and light chain variable regions of the antibody are derived from a first antibody (e.g., a murine antibody) and the heavy and light chain constant regions of the antibody are derived from a second antibody (e.g., a human antibody).

The term “humanized antibody” herein refers to a genetically engineered non-human antibody that has an amino acid sequence modified to increase the homology to the sequence of a human antibody. Generally, all or part of the CDRs of a humanized antibody are derived from a non-human antibody (donor antibody), and all or part of the non-CDRs (e.g., FRs and/or constant regions in variable regions) are derived from a human immunoglobulin (receptor antibody). The humanized antibody generally retains or partially retains the desired properties of the donor antibody, including, but not limited to, antigen specificity, affinity, reactivity, the ability to increase the activity of immune cells, the ability to enhance immune response, and the like.

The term “fully human antibody” herein refers to an antibody having variable regions in which both the FRs and CDRs are derived from human germline immunoglobulin sequences. Furthermore, if the antibody comprises constant regions, the constant regions are also derived from human germline immunoglobulin sequences. The “fully human antibody” herein may include amino acid residues that are not encoded by human germline immunoglobulin sequences (e.g., mutations introduced by random or site-specific mutagenesis in vitro or by somatic mutations in vivo). However, “fully human antibody” herein does not include antibodies in which CDR sequences derived from the germline of another mammalian species (e.g., mouse) have been grafted onto human framework sequences.

The term “naked antibody” herein refers to an antibody that is not linked, fused, or conjugated to another agent or molecule (e.g., a label or drug), peptide, or polypeptide. In specific embodiments, the naked antibody expressed by mammalian host cells can be glycosylated by the glycosylation machinery (e.g., glycosylases) of the host cells. In certain embodiments, the naked antibody is not glycosylated when expressed by a host cell that does not have its own glycosylation machinery (e.g., a glycosylase). In certain embodiments, the naked antibody is an intact antibody, while in other embodiments, the naked antibody is an antigen-binding fragment of an intact antibody.

The term “variable region” herein refers to a region of a heavy or light chain of an antibody involved in the binding of the antibody to an antigen. The “heavy chain variable region” is used interchangeably with “VH” and “HCVR”, and the “light chain variable region” is used interchangeably with “VL” and “LCVR”. Heavy and light chain variable domains (VH and VL, respectively) of natural antibodies generally have similar structures, each of which contains four conservative framework regions (FRs) and three hypervariable regions (HVRs). A single VH or VL domain may be sufficient to provide antigen-binding specificity. The terms “complementarity determining region” and “CDR” herein are used interchangeably and generally refer to a hypervariable region (HVR) of a heavy chain variable region (VH) or a light chain variable region (VL), which is also known as the complementarity determining region as it is precisely complementary to an antigenic epitope in spatial structure, wherein the heavy chain variable region CDR may be abbreviated as HCDR and the light chain variable region CDR may be abbreviated as LCDR. The terms “framework region” and “FR” are used interchangeably and refer to those amino acid residues of an antibody heavy chain variable region or light chain variable region other than CDRs. Generally, a typical antibody variable region consists of 4 FRs and 3 CDRs in the following order: FR1-CDR1-FR2-CDR2-FR3-CDR3-FR4.

The “CDR” herein may be labeled and defined in a manner well known in the art, including but not limited to the Kabat numbering scheme, Chothia numbering scheme, or IMGT numbering scheme; the tool websites used include, but are not limited to, the AbRSA site (http://cao.labshare.cn/AbRSA/cdrs.php), ab Ysis site (www.abysis.org/abysis/sequence_input/key_annotation/key_annotation.cgi), and IMGT site (http://www.imgt.org/3Dstructure-DB/cgi/DomainGapAlign.cgi#results). The CDR herein includes overlaps and subsets of amino acid residues defined in different ways.

The term “Kabat numbering scheme” herein generally refers to the immunoglobulin alignment and numbering scheme proposed by Elvin A. Kabat (see, e.g., Kabat et al., Sequences of Proteins of Immunological Interest, 5th Ed. Public Health Service, National Institutes of Health, Bethesda, Md., 1991).

The term “Chothia numbering scheme” herein generally refers to the immunoglobulin numbering scheme proposed by Chothia et al., which is a classical rule for identifying CDR region boundaries based on the position of structural loop regions (see, e.g., Chothia & Lesk (1987) J. Mol. Biol. 196: 901-917; Chothia et al., (1989) Nature 342: 878-883).

The term “IMGT numbering scheme” herein generally refers to a numbering scheme based on the international ImMunoGeneTics information system (IMGT) initiated by Lefranc et al. See Lefranc et al., Dev. Comparat. Immunol. 27:55-77, 2003.

The term “heavy chain constant region” herein refers to the carboxyl-terminal portion of an antibody heavy chain that is not directly involved in the binding of the antibody to an antigen, but exhibits effector functions, such as interaction with an Fc receptor; the heavy chain constant region has a more conserved amino acid sequence relative to the variable domain of the antibody. The “heavy chain constant region” at least comprises: a CH1 domain, a hinge region, a CH2 domain, a CH3 domain, or a variant or fragment thereof. The “heavy chain constant region” includes a “full-length heavy chain constant region” having a structure substantially similar to that of a natural antibody constant region and a “heavy chain constant region fragment” including only “a portion of the full-length heavy chain constant region”. Illustratively, a typical “full-length antibody heavy chain constant region” consists of the CH1 domain-hinge region-CH2 domain-CH3 domain. When the antibody is IgE, it further comprises a CH4 domain; when the antibody is a heavy-chain antibody, it does not comprise a CH1 domain. Illustratively, a typical “heavy chain constant region fragment” may be selected from CH1, Fc, and CH3 domains.

The term “light chain constant region” herein refers to the carboxyl-terminal portion of an antibody light chain that is not directly involved in the binding of the antibody to an antigen. The light chain constant region may be selected from a constant k domain and a constant 2 domain.

The term “Fc” herein refers to the carboxyl-terminal portion of an antibody that is formed by the hydrolysis of an intact antibody by papain, and the Fc region includes, for example, an Fc region of native sequences, a recombinant Fc region, and a variant Fc region. Although the boundaries of the Fc region of an immunoglobulin heavy chain may vary slightly, the human IgG heavy chain Fc region is generally defined to stretch from an amino acid residue at position Cys226, or from Pro230, to the carboxyl terminus thereof. The C-terminal lysine (residue 447 according to the Kabat numbering scheme) of the Fc region may be removed, for example, during the production or purification of the antibody, or by recombinant engineering of a nucleic acid encoding the heavy chain of the antibody, and thus, the Fc region may or may not comprise C-terminal lysine (Lys447), or C-terminal glycine (Gly446) and lysine (Lys447). Typically, the IgG Fc region comprises IgG CH2 and IgG CH3 domains; optionally, the IgG Fc region may further comprise a complete or partial hinge region, but does not comprise a CH1 domain. The “CH2 domain” of the human IgG Fc region typically extends from an amino acid residue at about position 231 to an amino acid residue at about position 340. In one embodiment, a carbohydrate chain is attached to the CH2 domain. The CH2 domain herein may be a CH2 domain of a native sequence or a variant CH2 domain. The “CH3 domain” comprises the residues in the Fc region at the C-terminus of the CH2 domain (i.e., from an amino acid residue at about position 341 to an amino acid residue at about position 447 of the IgG). The CH3 region herein may be a CH3 domain of a native sequence or a variant CH3 domain (e.g., a CH3 domain having a “protuberance” (“knob”) introduced in one strand and a “cavity” (“hole”) correspondingly introduced in the other strand; see U.S. Pat. No. 5,821,333, which is explicitly incorporated herein by reference). As described herein, such variant CH3 domains may be used to promote the heterodimerization of two non-identical antibody heavy chains.

Unless otherwise specified herein, the numbering of amino acid residues in the Fc region or constant region conforms to the EU numbering scheme, also known as the EU index, as described in Kabat et al., Sequences of Proteins of Immunological Interest, 5th Ed., Public Health Service, National Institutes of Health, Bethesda, MD, 1991.

The term “Fc variant” herein refers to a change in the structure or functionality of an Fc caused by the presence of one or more amino acid substitution, insertion, or deletion mutations at appropriate sites on the Fc. The “inter-Fc variant interaction” refers to the formation of space-filling effects, electrostatic steering, hydrogen bonding, hydrophobic interactions, etc., between mutationally designed Fc variants. The inter-Fc variant interaction contributes to the formation of stable heterodimeric proteins. Preferred mutational designs are those of the “knob-into-hole (KIH)” format. Techniques for designing mutations in Fc variants have been widely used in the art to prepare bispecific antibodies or heterodimeric Fc fusion protein forms. Representative examples include the “knob-into-hole” format proposed by Cater et al. (Ridgway, J. B., Presta, L. G., & Carter, P. (1996). ‘Knobs-into-holes’ engineering of antibody CH3 domains for heavy chain heterodimerization. Protein Engineering, Design and Selection, 9(7), 617-621.); the use of electrostatic steering to form a Fc-containing heterodimer format by Amgen technicians (US20100286374 A1); the IgG/IgA strand exchange used to form a heterodimer format (SEEDbodies) proposed by Jonathan H. Davis et al. (Davis, J. H., Aperlo, C., Li, Y., Kurosawa, E., Lan, Y., Lo, K. M., & Huston, J. S. (2010). SEEDbodies: fusion proteins based on strand-exchange engineered domain (SEED) CH3 heterodimers in an Fc analogue platform for asymmetric binders or immunofusions and bispecific antibodies. Protein Engineering, Design & Selection, 23 (4), 195-202.); the Genmab's DuoBody platform technology used to form bispecific molecules (Gramer, M. J., van den Bremer, E. T., van Kampen, M. D., Kundu, A., Kopfmann, P., Etter, E., & Parren, P. W. (2013 November). Production of stable bispecific IgG1 by controlled Fab-arm exchange: scalability from bench to large-scale manufacturing by application of standard approaches. In MAbs (Vol. 5, No. 6, pp. 962-973). Taylor & Francis.); the integration of structural calculation and Fc amino acid mutations, together with the integration of different modes of action, used to form the heterodimeric protein format by Xencor technicians (Moore, G. L., Bautista, C., Pong, E., Nguyen, D. H. T., Jacinto, J., Eivazi, A., & Lazar, G. A. (2011 November). A novel bispecific antibody format enables simultaneous bivalent and monovalent co-engagement of distinct target antigens. In MAbs (Vol. 3, No. 6, pp. 546-557). Taylor & Francis.); the charge network-based Fc modification method used to obtain the heterodimeric protein format by Suzhou Alphamab Co., Ltd. (CN201110459100.7); and other genetic engineering methods based on Fc amino acid variations or functional modifications to achieve the formation of functional heterodimeric proteins. The knob/hole structures on the Fc variant fragment described in the present disclosure refer to that the two Fc fragments are mutated separately and can be bound by the “knob-into-hole” format after mutation. The “knob-into-hole” model of Cater et al. is preferred for site-directed mutagenesis on the Fc region, such that the resulting first and second Fc variants can bind together in the “knob-into-hole” form to give a heterodimer. The selection of a particular immunoglobulin Fc region from a particular immunoglobulin class and subclass is within the knowledge of those skilled in the art. Preferred are the Fc regions of human IgG1, IgG2, IgG3, or IgG4 antibodies, and more preferred is the Fc region of human IgG1 antibody. One of the first Fc variant or the second Fc variant is randomly selected for the knob mutation and the other for hole mutation.

The term “effector function” herein refers to the activity of an antibody molecule that is mediated by binding through a domain rather than the antigen-binding domain of the antibody, typically mediated by binding of an effector molecule. Effector functions include complement-mediated effector functions, which are mediated by, for example, the binding of the C1 component of the complement to the antibody. Activation of a complement is important in the opsonization and lysis of cellular pathogens. Activation of a complement also stimulates inflammatory responses and may be involved in autoimmune hypersensitivity responses. Effector functions also include Fc receptor (FcR)-mediated effector functions, which may be triggered by the binding of the constant domain of an antibody to an Fc receptor (FcR). The binding of antibodies to Fc receptors on cell surfaces triggers many important and diverse biological responses, including the phagocytosis and destruction of antibody-coated particles, the clearance of immune complexes, the lysis of antibody-coated target cells by killer cells (known as antibody-dependent cell-mediated cytotoxicity, or ADCC), the release of inflammatory mediators, placental transfer, and the control of immunoglobulin production. The effector functions of an antibody can be altered by altering, such as enhancing or reducing, the affinity of the antibody for an effector molecule such as an Fc receptor or a complement component. Usually, the binding affinity will be altered by modifying the binding sites for effector molecules, and in this case, it is appropriate to locate the sites of interest and to modify at least a portion of the sites in a suitable manner. It is also contemplated that alterations to the binding site on an antibody for an effector molecule need not significantly alter the overall binding affinity but may alter the geometry of the interaction, thereby rendering the effector mechanism ineffective, as in non-productive binding. It is further contemplated that effector functions may also be altered by modifying sites that are not directly involved in effector molecule binding but are otherwise involved in the performance of effector functions.

The term “epitope” herein includes any protein determinant capable of specifically binding to an immunoglobulin, an scFv, or a T-cell receptor. The term “epitope” includes any protein determinant capable of specifically binding to an immunoglobulin or a T-cell receptor. Epitopic determinants usually consist of chemically active surface clusters of molecules such as amino acids or sugar side chains and usually have specific three-dimensional structural characteristics, as well as specific charge characteristics. For example, antibodies may be produced against N-terminal or C-terminal peptides of a polypeptide.

The term “mutation” herein includes gene mutations and amino acid mutations, wherein gene mutations refer to deletions, or insertions, inversions, or substitutions of heterologous nucleic acids, which may result in changes in the amino acid sequence of the corresponding protein product; amino acid mutations, also known as non-synonymous single nucleotide mutations, result in changes in the amino acid sequence of the protein product due to changes in some single bases. The amino acid changes can affect protein stability, interaction, and enzyme activity, thereby leading to the development of diseases.

The term “amino acid (aa)” herein is a basic unit that constitutes a protein, imparting a specific molecular structural morphology to the protein so that the molecule has biochemical activity. In chemistry, an amino acid refers to an organic compound that contains an amino group (—NH2) and a carboxyl group (—COOH) in the structure. Amino acids can be classified into α, β, γ, δ, etc. amino acids according to the position of the carbon atom to which the amino group is linked in the carboxylic acid: the amino group and the carboxyl group of an a amino acid are linked to the same carbon atom, the amino group and the carboxyl group of a β amino acid are linked to adjacent carbon atoms, and so on. In biology, amino acids generally refer to a amino acids, that is, amino acids in which an amino group and a carboxyl group are directly linked to the same-CH-structure, and the general formula is H2NCHRCOOH (R represents a certain organic substituent). Illustratively, the common 20 amino acids include glycine, alanine, valine, leucine, isoleucine, phenylalanine, tryptophan, tyrosine, aspartate, histidine, asparagine, glutamate, lysine, glutamine, methionine, arginine, serine, threonine, cysteine, and proline.

The term “amino acid substitution” herein refers to those in which at least one amino acid residue in a native or starting sequence is removed and a different amino acid is inserted in its place at the same position. Substitutions may be single in which only one amino acid in the molecule has been substituted, or they may be multiple in which two or more amino acids in the same molecule have been substituted.

The term “conservative amino acid substitution” herein refers to the substitution of an amino acid that is typically present in a sequence with a different amino acid of similar size, charge, or polarity. Examples of conservative substitutions include the substitution of a non-polar (hydrophobic) residue such as isoleucine, valine, and leucine with another non-polar residue. Likewise, examples of conservative substitutions include the substitution of one polar (hydrophilic) residue with another residue, such as between arginine and lysine, between glutamine and asparagine, and between glycine and serine. In addition, substitution of a basic residue such as lysine, arginine, or histidine with another residue, or substitution of one acidic residue such as aspartate or glutamate with another acidic residue is additional examples of conservative substitutions. Examples of non-conservative substitutions include the substitution of a non-polar (hydrophobic) amino acid residue such as isoleucine, valine, leucine, alanine, or methionine with a polar (hydrophilic) residue such as cysteine, glutamine, glutamate, or lysine, and/or the substitution of a polar residue with a non-polar residue. The term “mutant” herein means that a “variant” of the protein or peptide may have at least 70%, 75%, 80%, 85%, 90%, 95%, 98%, or 99% amino acid identity to the amino acid sequence of the protein or peptide.

The term “nucleic acid” herein includes any compound and/or substance that comprises a polymer of nucleotides. Each nucleotide consists of a base, in particular a purine or pyrimidine base (i.e., cytosine (C), guanine (G), adenine (A), thymine (T), or uracil (U)), a sugar (i.e., deoxyribose or ribose), and a phosphate group. Generally, a nucleic acid molecule is described as a sequence of bases, whereby the bases represent the primary structure (linear structure) of the nucleic acid molecule. The sequence of bases is generally expressed as 5′ to 3′. Herein, the term “nucleic acid molecule” encompasses deoxyribonucleic acid (DNA), including, e.g., complementary DNA (cDNA) and genomic DNA; ribonucleic acid (RNA), in particular messenger RNA (mRNA); the synthetic forms of DNA or RNA; and polymers comprising a mixture of two or more of these molecules. The nucleic acid molecule may be linear or cyclic. Furthermore, the term “nucleic acid molecule” includes both sense and antisense strands, as well as single- and double-stranded forms. Moreover, the nucleic acid molecules described herein may contain naturally occurring or non-naturally occurring nucleotides. Examples of non-naturally occurring nucleotides include modified nucleotide bases having derived sugar or phosphate backbone bonding or chemically modified residues. The nucleic acid molecule also encompasses DNA and RNA molecules suitable for use as vectors for direct expression of the antibodies of the present disclosure in vitro and/or in vivo, e.g., in a host or patient. Such DNA (e.g., cDNA) or RNA (e.g., mRNA) vectors may be unmodified or modified. For example, mRNA can be chemically modified to enhance the stability of the RNA vector and/or the expression of the encoded molecule such that the mRNA can be injected into a subject to produce antibodies in vivo.

The term “identity” herein can be obtained by calculating as follows: to determine the percent “identity” of two amino acid sequences or two nucleic acid sequences, the sequences are aligned for optimal comparison purposes (e.g., for optimal alignment, gaps can be introduced in one or both of the first and second amino acid sequences or nucleic acid sequences, or non-homologous sequences can be discarded for comparison purpose). Amino acid residues or nucleotides at corresponding amino acid positions or nucleotide positions are then compared. When a position in the first sequence is occupied by the same amino acid residue or nucleotide at the corresponding position in the second sequence, the molecules are identical at this position.

The percent identity between two sequences varies with the identical positions shared by the sequences, taking into account the number of gaps that need to be introduced and the length of each gap for optimal alignment of the two sequences.

A mathematical algorithm can be used to compare two sequences and calculate the percent identity between the sequences. For example, the percent identity between two amino acid sequences is determined with the Needleman and Wunsch algorithm ((1970) J. Mol. Biol., 48: 444-453; available at www.gcg.com) which has been integrated into the GAP program of the GCG software package, using the Blosum 62 matrix or PAM250 matrix and gap weight of 16, 14, 12, 10, 8, 6, or 4 and length weight of 1, 2, 3, 4, 5, or 6. For another example, the percent identity between two nucleotide sequences is determined with the GAP program of the GCG software package (available at www.gcg.com), using the NWSgapdna.CMP matrix and gap weight of 40, 50, 60, 70, or 80 and length weight of 1, 2, 3, 4, 5, or 6. A particularly preferred parameter set (and one that should be used unless otherwise stated) is a Blosum62 scoring matrix with a gap penalty of 12, a gap extension penalty of 4, and a frameshift gap penalty of 5.

The percent identity between two amino acid sequences or nucleotide sequences can also be determined with a PAM120 weighted remainder table, a gap length penalty of 12, and a gap penalty of 4, using the E. Meyers and W. Miller algorithm ((1989) CABIOS, 4: 11-17) which has been incorporated into the ALIGN program (version 2.0).

Additionally or alternatively, the nucleic acid sequences and protein sequences described in the present disclosure can be further used as “query sequences” for searches against public databases to, e.g., identify sequences of other family members or correlated sequences. For example, such searches can be performed using the NBLAST and XBLAST programs (version 2.0) of Altschul et al., (1990) J. Mol. Biol., 215: 403-10. BLAST nucleotide searches can be performed with the NBLAST program. BLAST protein searches can be performed with the XBLAST program to obtain amino acid sequences homologous to the protein molecule of the present disclosure. To obtain gapped alignment results for the purpose of comparison, gapped BLAST can be used as described in Altschul et al., (1997) Nucleic Acids Res. 25: 3389-3402. When using the BLAST and gapped BLAST programs, the default parameters of the respective programs (e.g., XBLAST and NBLAST) can be used. See www.ncbi.nlm.nih.gov.

The term “chimeric antigen receptor (CAR)” herein refers to an artificial cell surface receptor engineered to express on an immune effector cell and specifically bind to an antigen, which comprises at least (1) an extracellular antigen-binding domain, e.g., a variable heavy or light chain of an antibody, (2) a transmembrane domain that anchors the CAR into the immune effector cell, and (3) an intracellular signaling domain. The CAR is capable of redirecting T cells and other immune effector cells to a selected target, e.g., a cancer cell, in a non-MHC-restricted manner using the extracellular antigen-binding domain.

As used herein, the term “vector” includes nucleic acid vectors, e.g., DNA vectors (e.g., plasmids), RNA vectors, viruses, or other suitable replicons (e.g., viral vectors). Various vectors have been developed for the delivery of polynucleotides encoding foreign proteins into prokaryotic or eukaryotic cells. The expression vector of the present disclosure contains polynucleotide sequences as well as additional sequence elements, e.g., for expressing proteins and/or integrating these polynucleotide sequences into the genome of mammalian cells. Some vectors that may be used to express the antibody and antibody fragment of the present disclosure include plasmids comprising regulatory sequences (e.g., promoter and enhancer regions) that direct gene transcription. Other useful vectors for expressing the antibody and antibody fragment contain polynucleotide sequences that enhance the rate of translation of these genes or improve the stability or nuclear export of mRNA produced by gene transcription. These sequence elements include, for example, 5′ and 3′ untranslated regions, internal ribosome entry sites (IRESs), and polyadenylation signal sites, so as to direct the effective transcription of the gene carried on the expression vector. The expression vector of the present disclosure may also contain a polynucleotide encoding a marker for selecting cells comprising such a vector. Examples of suitable markers include genes encoding resistance to antibiotics (e.g., ampicillin, chloramphenicol, kanamycin, or nourseothricin).

The term “host cell” herein refers to a cell into which an exogenous nucleic acid has been introduced, including the progeny of such a cell. Host cells include “transformants” and “transformed cells”, which include primary transformed cells and progenies derived therefrom, regardless of the number of passages. Progenies may not be exactly the same as parent cells in terms of nucleic acid content, and may contain mutations. Mutant progenies having the same function or biological activity that are screened or selected from the primary transformed cells are included herein.

The term “pharmaceutical composition” herein refers to a formulation that exists in a form allowing the biological activity of the active ingredient contained therein to be effective and does not contain additional ingredients having unacceptable toxicity to a subject to which the pharmaceutical composition is administered.

The term “pharmaceutically acceptable excipient”, “pharmaceutically acceptable carrier”, or “pharmaceutically acceptable carrier, diluent, or adjuvant” herein refers to those excipients that do not have a significant irritating effect on an organism and do not impair the biological activity and properties of the active compound, and includes any and all solvents, dispersion media, coatings, surfactants, antioxidants, preservatives (e.g., antibacterial agents and antifungal agents), isotonic agents, absorption retardants, salts, preservatives, drug stabilizers, binders, excipients, disintegrants, lubricants, sweeteners, flavorants, dyes, and the like, and combinations thereof, as known to those skilled in the art (see, e.g., Remington's Pharmaceutical Sciences, 18th ED. MackPrinting Company, 1990, pp. 1289-1329). Except insofar as any conventional carrier is incompatible with the active ingredient, its use in the therapeutic or pharmaceutical compositions is contemplated.

The term “treatment” herein refers to surgical or therapeutic treatment for the purpose of preventing or slowing (reducing) the progression of an undesired physiological or pathological change, e.g., cancer, an immune-mediated inflammatory disease (IMID) (e.g., an autoimmune disease and an inflammatory disease), and virus infection, in a subject being treated. Beneficial or desired clinical results include, but are not limited to, alleviation of symptoms, decrease of severity of disease, stabilization (i.e., not worsening) of state of disease, delay or slowing of disease progression, amelioration or palliation of state of disease, and remission of state of disease (whether partial or total), whether detectable or undetectable. Subjects in need of treatment include those already with a disorder or disease, as well as those who are susceptible to a disorder or disease or those who intend to prevent a disorder or disease. When referring to terms such as slowing, alleviation, decrease, palliation, and remission, their meanings also include elimination, disappearance, nonoccurrence, etc.

The terms “subject” and “patient” herein refer to an organism that receives treatment for the particular disease or disorder described in the present disclosure. Examples of subjects and patients include mammals, such as humans, primates (e.g., monkeys), or non-primate mammals, that receive treatment for a disease or disorder. “Patient” also refers to an organism that receives treatment for a particular disease or disorder (e.g., an immune-mediated inflammatory disease (IMID) or a cancer) as described herein.

The term “effective amount” herein refers to an amount of a therapeutic agent that is effective to prevent or alleviate symptoms of a disease or the progression of the disease when administered to a cell, tissue, or subject alone or in combination with another therapeutic agent. “Effective amount” also refers to an amount of a compound that is sufficient to alleviate symptoms, e.g., to treat, cure, prevent, or alleviate related medical disorders, or to increase the rates at which such disorders are treated, cured, prevented, or alleviated. When the active ingredient is administered alone to an individual, a therapeutically effective dose refers to the amount of the ingredient alone. When a combination is used, a therapeutically effective dose refers to the combined amounts of the active ingredients that produce the therapeutic effect, whether administered in combination, sequentially, or simultaneously.

The term “immune-mediated inflammatory disease” or “IMID” herein refers to any group of disorders or diseases that lack a clear etiology but are characterized by common inflammatory pathways leading to inflammation, and which may result from or be triggered by dysregulation of a normal immune response. The “immune-mediated inflammatory disease” herein encompasses autoimmune diseases and inflammatory diseases.

“Autoimmune disease” herein refers to a disorder of cellular, tissue, and/or organ damage resulting from an immune response in a subject to its own cells, tissues, and/or organs. The “inflammatory disease” herein refers to a disorder in a subject characterized by inflammation, preferably chronic inflammation. Autoimmune disorders may or may not be accompanied by inflammation. Furthermore, the inflammation may or may not be caused by an autoimmune disorder. Herein, the terms “autoimmune disease” and “autoimmune-related disease” are used interchangeably.

In some embodiments, exemplary immune-mediated inflammatory diseases include: inflammatory bowel disease (e.g., Crohn's disease and ulcerative colitis), multiple sclerosis, psoriasis, rheumatoid arthritis, psoriatic arthritis, fibrosis, transplant rejection, graft-versus-host disease, ankylosing spondylitis, arteriosclerosis, rheumatism, scleroderma, uveitis, and the like.

The term “cancer” herein refers to or describes a physiological condition in mammals that is typically characterized by unregulated cell growth. Included in this definition are benign and malignant cancers. The term “tumor” or “neoplasm” herein refers to all neoplastic cell growth and proliferation, whether malignant or benign, and all pre-cancerous and cancerous cells and tissues. The terms “cancer” and “tumor” are not mutually exclusive when referred to herein.

### BRIEF DESCRIPTION OF THE DRAWINGS

FIG. 1 shows possible formats of bispecific molecules combinations.

FIG. 2 shows the binding activity of the bispecific molecules to the human TL1A protein.

FIG. 3 shows the effect of the bispecific molecules on blocking the interaction between human TL1A and CHO-K1-human DR3 cells.

FIG. 4 shows the effect of the bispecific molecules on blocking the interaction between human TL1A and the DcR3 protein.

FIG. 5 shows the inhibition of NFκB activity in TF-1-NFκB-luciferase cells by the bispecific molecules.

FIG. 6 shows the inhibition of apoptotic activity of TF-1 cells by the bispecific molecules.

FIG. 7 shows the inhibition of IFN-γ secretion from PBMCs stimulated with human TL1A, IL-12, and IL-18 by the bispecific molecules.

FIG. 8 shows the binding activity of the bispecific molecules to the human IL23 protein.

FIG. 9 shows the effect of the bispecific molecules on blocking the interaction between human IL23 and IL23R.

FIG. 10 shows the effect of the bispecific molecules on blocking the interaction between human IL23 and CHO-K1-human IL23R.

FIG. 11 shows the inhibition of human IL23-induced downstream signaling of HEK293-IL23R-STAT3 reporter gene cells by the bispecific molecules.

FIG. 12 shows the inhibition of human IL23-induced mIL 17 factor release in mouse spleen by the bispecific molecules.

FIG. 13 shows the simultaneous binding activity of the bispecific molecules to TL1A and IL23 proteins.

FIG. 14 shows the synergistic inhibition of the TL1A- and IL23-induced mouse Th17 differentiation by the bispecific molecules.

FIG. 15-16 shows the effect of bispecific molecules on mouse body weight in a cytokine-induced, mouse model of Inflammatory Bowel Disease (IBD).

### DETAILED DESCRIPTION

The present disclosure is further described below with reference to specific examples; the advantages and features of the present disclosure will become more apparent with the description. Experimental procedures without specified conditions in the examples are conducted according to conventional conditions or conditions recommended by the manufacturers. Reagents or instruments without specified manufacturers used herein are conventional products that are commercially available.

The examples herein are illustrative only, and do not limit the scope of the present disclosure in any way. It will be appreciated by those skilled in the art that various modifications or substitutions may be made to the technical solutions of the present disclosure in form and details without departing from the spirit and scope of the present disclosure, and that these modifications and substitutions shall fall within the protection scope of the present disclosure.

### Example 1. Construction of Recombinant Proteins and Engineered Cell Strains

### 1.1 Preparation of Recombinant Human TL1A and Mouse TL1A Proteins

The amino acid sequences of the antigens and the proteins for detection involved in the present disclosure were designed by using the extracellular region of the human TL1A protein (UniProt sequence No.: O95150, SEQ ID NO: 1) and the extracellular region of the mouse TL1A protein (UniProt sequence No.: Q5UBV8, SEQ ID NO: 2) as templates of TL1A of the present disclosure (Table 1). Optionally, different tags (e.g., his tag) were fused on the basis of the TL1A proteins. The resulting sequences were cloned into a PTT5 vector (Invitrogen), respectively, and transiently expressed in 293 cells or stably expressed and purified in CHO cells to obtain the encoded antigens and the proteins for detection of the present disclosure.

TABLE 1
Sequences of TL1A protein templates

| Sequence | Sequence | |
|---|---|---|
| name | No. | Amino acid sequence |
| Human TL1A | 1 | LKGQEFAPSHQQVYAPLRADGDKPRAHLTVVRQTPTQHFKNQFPALHWEHE |
| extracellular | | LGLAFTKNRMNYTNKFLLIPESGDYFIYSQVTFRGMTSECSEIRQAGRPNKPDS |
| region | | ITVVITKVTDSYPEPTQLLMGTKSVCEVGSNWFQPIYLGAMFSLQEGDKLMV |
| | | NVSDISLVDYTKEDKTFFGAFLL |
| Mouse TL1A | 2 | AGQLRVPGKDCMLRAITEERSEPSPQQVYSPPRGKPRAHLTIKKQTPAPHLKN |
| extracellular | | QLSALHWEHDLGMAFTKNGMKYINKSLVIPESGDYFIYSQITFRGTTSVCGDI |
| region | | SRGRRPNKPDSITVVITKVADSYPEPARLLTGSKSVCEISNNWFQSLYLGAMFS |
| | | LEEGDRLMVNVSDISLVDYTKEDKTFFGAFLL |

### 1.2 Preparation of Human IL23 Protein and Mouse IL23 Protein

The amino acid sequences of the antigens and the proteins for detection involved in the present disclosure were designed by using human IL23 (human IL23A subunit, UniProt sequence No.: Q9NPF7 (Arg20-Pro189), human IL12B subunit, UniProt sequence No.: P29460 (Ile23-Ser328)), mouse IL23 (mouse IL23A subunit, UniProt sequence No.: Q9EQ14-1 (Val22-Ala196), and mouse IL12B subunit, UniProt sequence No.: P43432 (Met23-Ser335)) as templates of the present disclosure. Optionally, a his-avi-biotin or his tag was fused on the basis of the human IL23 protein and the mouse IL23 protein. The resulting sequences were cloned into a PTT5 vector (Invitrogen), respectively, and transiently expressed in Expi293 cells. After 7 days of cell culture, the cells were removed by filtration, and the supernatant was collected and subjected to affinity purification with a Ni filler. The protein was eluted stepwise with imidazole at different concentrations, and the target sample was recovered. Aggregates could be further removed using gel filtration chromatography or anion exchange.

TABLE 2
Sequences of IL23 protein templates

| | Sequence | |
|---|---|---|
| Sequence name | No. | Amino acid sequence |
| Human IL23A | 3 | RAVPGGSSPAWTQCQQLSQKLCTLAWSAHPLVGHMDLREEGDEETTNDVP |
| | | HIQCGDGCDPQGLRDNSQFCLQRIHQGLIFYEKLLGSDIFTGEPSLLPDSPVG |
| | | QLHASLLGLSQLLQPEGHHWETQQIPSLSPSQPWQRLLLRFKILRSLQAFVA |
| | | VAARVFAHGAATLSP |
| Human IL12B | 4 | IWELKKDVYVVELDWYPDAPGEMVVLTCDTPEEDGITWTLDQSSEVLGSG |
| | | KTLTIQVKEFGDAGQYTCHKGGEVLSHSLLLLHKKEDGIWSTDILKDQKEP |
| | | KNKTFLRCEAKNYSGRFTCWWLTTISTDLTFSVKSSRGSSDPQGVTCGAAT |
| | | LSAERVRGDNKEYEYSVECQEDSACPAAEESLPIEVMVDAVHKLKYENYTS |
| | | SFFIRDIIKPDPPKNLQLKPLKNSRQVEVSWEYPDTWSTPHSYFSLTFCVQVQ |
| | | GKSKREKKDRVFTDKTSATVICRKNASISVRAQDRYYSSSWSEWASVPCS |
| Mouse IL23A | 5 | VPRSSSPDWAQCQQLSRNLCMLAWNAHAPAGHMNLLREEEDEETKNNVP |
| | | RIQCEDGCDPQGLKDNSQFCLQRIRQGLAFYKHLLDSDIFKGEPALLPDSPM |
| | | EQLHTSLLGLSQLLQPEDHPRETQQMPSLSSSQQWQRPLLRSKILRSLQAFL |
| | | AIAARVFAHGAATLTEPLVPTA |
| Mouse IL23B | 6 | MWELEKDVYVVEVDWTPDAPGETVNLTCDTPEEDDITWTSDQRHGVIGSG |
| | | KTLTITVKEFLDAGQYTCHKGGETLSHSHLLLHKKENGIWSTEILKNFKNKT |
| | | FLKCEAPNYSGRFTCSWLVQRNMDLKFNIKSSSSSPDSRAVTCGMASLSAE |
| | | KVTLDQRDYEKYSVSCQEDVTCPTAEETLPIELALEARQQNKYENYSTSFFI |
| | | RDIIKPDPPKNLQMKPLKNSQVEVSWEYPDSWSTPHSYFSLKFFVRIQRKKE |
| | | KMKETEEGCNQKGAFLVEKTSTEVQCKGGNVCVQAQDRYYNSSCSKWAC |
| | | VPCRVRS |

### 1.3 Preparation of CHO-K1 Human DR3 Cells

A corresponding nucleotide sequence encoding a full-length amino acid sequence of human DR3 (UniProt sequence No.: Q93038, SEQ ID NO: 7) (Table 3) was cloned into a pcDNA3.1 vector (purchased from Clontech), and a plasmid was prepared. After plasmid transfection (Lipofectamine® 3000 Transfection Kit, purchased from Invitrogen, Cat. No.: L3000-015) into the CHO-K1 cell line (purchased from the Chinese Academy of Sciences), the cells were selectively cultured in a DMEM/F12 medium containing 10 μg/mL puromycin and 10% (w/w) fetal bovine serum for 2 weeks, and then monoclonal cells were plated in a 96-well plate and cultured at 37° C. with 5% (v/v) CO₂. After about 2 weeks, some of the monoclonal wells were selected for expansion. The expanded clones were screened by flow cytometry. Monoclonal cell lines with better growth and higher fluorescence intensity were selected for further expansion and cryopreserved in liquid nitrogen.

TABLE 3
Full-length amino acid sequence of human DR3

| Sequence | Sequence | |
|---|---|---|
| name | No. | Amino acid sequence |
| Human DR3 | 7 | MEQRPRGCAAVAAALLLVLLGARAQGGTRSPRCDCAGDFHKKIGLFCC |
| | | RGCPAGHYLKAPCTEPCGNSTCLVCPQDTFLAWENHHNSECARCQACD |
| | | EQASQVALENCSAVADTRCGCKPGWFVECQVSQCVSSSPFYCQPCLDCG |
| | | ALHRHTRLLCSRRDTDCGTCLPGFYEHGDGCVSCPTSTLGSCPERCAAV |
| | | CGWRQMFWVQVLLAGLVVPLLLGATLTYTYRHCWPHKPLVTADEAGM |
| | | EALTPPPATHLSPLDSAHTLLAPPDSSEKICTVQLVGNSWTPGYPETQEAL |
| | | CPQVTWSWDQLPSRALGPAAAPTLSPESPAGSPAMMLQPGPQLYDVMD |
| | | AVPARRWKEFVRTLGLREAEIEAVEVEIGRFRDQQYEMLKRWRQQQPA |
| | | GLGAVYAALERMGLDGCVEDLRSRLQRGP |

### 1.4 Preparation of TF-1-NFκB-Luciferase Cells

After plasmid transfection (Lipofectamine® 3000 Transfection Kit, purchased from Invitrogen, Cat. No.: L3000-015) of the NF-kBre-nanoLuc plasmid (purchased from Promega) into the TF-1 cell line (Cobioer, Cat. No.: CBP60808), the cells were selectively cultured in a 1640 medium containing 1 μg/mL puromycin and 10% (w/w) fetal bovine serum for 2 weeks, and then monoclonal cells were plated in a 96-well plate and cultured at 37° C. with 5% (v/v) CO₂. After about 2 weeks, some of the monoclonal wells were selected for expansion. The expanded clones were screened by flow cytometry. Monoclonal cell lines with better growth and higher reporter gene signals were selected for further expansion and cryopreserved in liquid nitrogen.

### 1.5 Preparation of CHO-K1 Human IL23R Cells

A corresponding nucleotide sequence encoding a full-length amino acid sequence of human IL23R (UniProt sequence No.: Q5VWK5, SEQ ID NO: 8) (Table 4) was cloned into a pLVX vector (purchased from Clontech), and a plasmid was prepared. The pLVX-human IL23R plasmid was subjected to lentiviral coating and then was used for the lentiviral infection of CHO-K1 cells (purchased from the Chinese Academy of Sciences). After the cells were selectively cultured in a DMEM/F12 medium containing 10 μg/mL puromycin, 4 mM Glutamine, and 10% (w/w) fetal bovine serum for 1 week, the expanded cells were assayed by flow cytometry, further expanded, and cryopreserved in liquid nitrogen.

TABLE 4
Full-length amino acid sequence of human IL23R

| Sequence | Sequence | |
|---|---|---|
| name | No. | Amino acid sequence |
| Human | 8 | MNQVTIQWDAVIALYILFSWCHGGITNINCSGHIWVEPATIFKMGMNISIY |
| IL23R | | CQAAIKNCQPRKLHFYKNGIKERFQITRINKTTARLWYKNFLEPHASMYC |
| | | TAECPKHFQETLICGKDISSGYPPDIPDEVTCVIYEYSGNMTCTWNAGKL |
| | | TYIDTKYVVHVKSLETEEEQQYLTSSYINISTDSLQGGKKYLVWVQAAN |
| | | ALGMEESKQLQIHLDDIVIPSAAVISRAETINATVPKTIIYWDSQTTIEKVS |
| | | CEMRYKATTNQTWNVKEFDTNFTYVQQSEFYLEPNIKYVFQVRCQETG |
| | | KRYWQPWSSLFFHKTPETVPQVTSKAFQHDTWNSGLTVASISTGHLTSD |
| | | NRGDIGLLLGMIVFAVMLSILSLIGIFNRSFRTGIKRRILLLIPKWLYEDIPN |
| | | MKNSNVVKMLQENSELMNNNSSEQVLYVDPMITEIKEIFIPEHKPTDYKK |
| | | ENTGPLETRDYPQNSLFDNTTVVYIPDLNTGYKPQISNFLPEGSHLSNNNE |
| | | ITSLTLKPPVDSLDSGNNPRLQKHPNFAFSVSSVNSLSNTIFLGELSLILNQ |
| | | GECSSPDIQNSVEEETTMLLENDSPSETIPEQTLLPDEFVSCLGIVNEELPSI |
| | | NTYFPQNILESHENRISLLEK |

### Example 2. Sequence Design of Antibodies

### 2.1 Sequence Design of Anti-TL1A Antibodies

The anti-TL1A antibodies used in the present disclosure include RVT3101 (publication No. CN113150144A), as well as the in-house developed anti-TL1A monoclonal antibodies Hu001_L3H3 and Hu004_L1H1, and the anti-TL1A single domain antibodies HcAb5-H5, HcAb7-H5c, and HcAb8-H4a. The sequences of the TL1A antibodies are shown in Table 6, and the Kabat analysis of the CDR sequences is shown in Table 7.

### 2.2 Sequence Design of Anti-IL23 Antibodies

The sequence of the anti-IL23 antibody used in the present disclosure is selected from the TREMFYA (Guselkumab) sequence of Janssen (Pat. No. CN101389351B), SKYRIZI (Risankizumab) of Abb Vie (Pat. No. CN103282382B), and nanobody 37D5 against the p19 subunit of hIL-23 (Aline D, Silvia S, Carlo B, et al., Front Immunol, 2017, 8:884; Pat. No. WO2009068627A2).

Meanwhile, mutation design was performed internally on the variable regions of the parent antibody (Guselkumab), including prediction of a complex based on the parent antibody and IL-23p19, analysis of the amino acids involved in interfacial interactions within the three-dimensional structure, and mutation of sites in CDR regions using various algorithms to maintain antibody activity. After analysis and identification, mutant antibodies PAb H7L8, PAb H3L5, and PAb H3L8 of the parent antibody were obtained. The sequences of the IL23 antibodies are shown in Table 6, and the Kabat analysis of the CDR sequences is shown in Table 7.

### 2.3 Sequence Design and Expression of Anti-TL1A×IL23p19 Bispecific Molecules

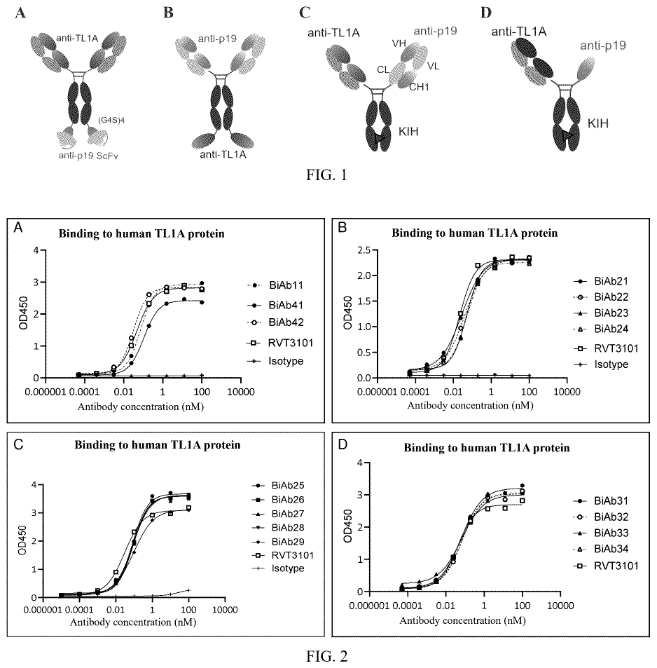

4 antibody structures were designed for anti-TL1A×IL23p19 bispecific molecules in the present disclosure, with structural schematic diagrams shown in FIG. 1, and the molecules are respectively the IgG-scFv antibody, the IgG-VHH bivalent antibody, the CrossMab antibody, and the Fab-VHH antibody. The bispecific molecules specifically bind to TL1A and IL23.

### A. Construction and Expression of IgG-scFv Antibody Format

The IgG-scFv antibody was constructed by linking the scFv fragment of another antibody to the C-termini of both heavy chains of an IgG antibody, and thus it comprises two heavy chains and two light chains. The two heavy chains comprise VH1-CH1-Fc-L1-VH2-L2-VL2, and the two light chains comprise VL1-CL; the L1 is (G4S) 4 (SEQ ID NO: 31), and the L2 is (G4S) 3 (SEQ ID NO: 32) (FIG. 1A).

### B. Construction and Expression of IgG-VHH Bivalent Antibody Format

The IgG-VHH antibody was constructed by linking the VHH fragment of another antibody to the C-termini of both heavy chains of an IgG antibody, and thus it comprises two heavy chains and two light chains. The heavy chains comprise VH1-CH1-Fc-L2-VH2, and the light chains comprise VL1-CL; and the L2 is (G4S) 3 (SEQ ID NO: 32) (FIG. 1B).

### C. Construction and Expression of CrossMab Antibody Format

The CrossMab bispecific antibody is an antibody employing a functional region exchange technology within the antibody Fab arms. Building upon the “knob-into-hole” technology, this technology resolves the issue of correct homologous light-heavy chain assembly through a functional region exchange within the Fab arms, thereby further increasing the success rate of assembly. The CrossMab bispecific antibody involved in the present disclosure comprises a first heavy chain and a second heavy chain, as well as a first light chain and a second light chain. The first heavy chain comprises VH1-CH1-Fc, the second heavy chain comprises VH2-CL-Fc, the first light chain comprises VL1-CL, and the second light chain comprises VL2-CH1 (FIG. 1C).

### D. Construction and Expression of Fab-VHH Antibody Format

The Fab-VHH hybrid antibody refers to an antibody whose variable region consists of an Fab and a single domain antibody, respectively, and comprises a first heavy chain, a second heavy chain, and one light chain, wherein the first heavy chain comprises VH1-CH1-Fc, the second heavy chain comprises VH2-Fc, and the light chain comprises VL1-CL (FIG. 1D).

The heavy chain and light chain cDNA sequences of the antibody were cloned into a PTT5 vector to obtain the heavy chain and light chain plasmids, respectively. The recombinant plasmids were co-transfected into Expi293F cells. After 7 days of cell culture, the cells were removed by filtration, and the supernatant was collected and subjected to affinity purification with a Protein A filler. The protein was eluted in one step with an Elution Buffer, and the target sample was recovered. Isomers, mispaired species, and/or aggregates could be further removed by methods such as gel filtration chromatography, hydrophobic chromatography, or mixed-mode chromatography (MMC) to obtain the purified bispecific molecule described above.

The structural composition of the bispecific molecules of the present disclosure is shown in Table 5, and the sequence information and the Kabat analysis of the CDR sequences are shown in Table 6 and Table 7, respectively.

TABLE 5
Structural composition of bispecific molecules

| | | | | | Heavy chain | Light chain |
|---|---|---|---|---|---|---|
| | | TL1A- | | | constant | constant |
| Antibody | Antibody | targeting | IL23-targeting | Linker | region | region |
| name | structure | moiety | moiety | fragment | subtype | subtype |
| BiAb11 | FIG. 1A | Hu004_L1H1 | Guselkumab VHVL | L1, L2 | IgG1 Fc1 | Kappa |
| BiAb21 | FIG. 1B | HcAb5-H5 | Guselkumab VHVL | L2 | IgG1 Fc2 | Lambda |
| BiAb22 | | | | | IgG1 Fc3 | |
| BiAb23 | | | Risankizumab VHVL | | IgG1 Fc2 | Kappa |
| BiAb24 | | | | | IgG1 Fc3 | |
| BiAb25 | | HcAb7-H5c | PAb H7L8 | | IgG1 Fc3 | Lambda |
| BiAb26 | | | PAb H3L5 | | | |
| BiAb27 | | | PAb H3L8 | | | |
| BiAb28 | | | Guselkumab VHVL | | | |
| BiAb29 | | HcAb8-H4a | Guselkumab VHVL | | | |
| BiAb31 | FIG. 1C | Hu001_L3H3 | Risankizumab VHVL | / | IgG1 KIH 1 | Kappa |
| BiAb32 | | Hu004_L1H1 | Guselkumab VHVL | / | IgG1 KIH | |
| BiAb33 | | Hu001_L3H3 | Guselkumab VHVL | / | 2(Knob 2/Hole 2) | |
| BiAb34 | | Hu001_L3H3 | Risankizumab VHVL | / | | |
| BiAb41 | FIG. 1D | HcAb5-H5 | Guselkumab VHVL | / | | Lambda |
| BiAb42 | | Hu004_L1H1 | 37D5V1 VH | / | | Kappa |

TABLE 6
Sequence information of bispecific molecules

| | Sequence | |
|---|---|---|
| Sequence name | No. | Amino acid sequence |
| RVT3101 VH | 9 | QVQLVQSGAEVKKPGASVKVSCKASGYDFTYYGISWVRQAPGQGL |
| | | EWMGWISTYNGNTHYARMLQGRVTMTTDTSTRTAYMELRSLRSDD |
| | | TAVYYCARENYYGSGAYRGGMDVWGQGTTVTVSS |
| RVT3101 VL | 10 | EIVLTQSPATLSLSPGERATLSCRASQSVSSYLAWYQQKPGQAPRLLI |
| | | YDASNRATGIPARFSGSGSGTDFTLTISSLEPEDFAVYYCQQRSNWP |
| | | WTFGQGTKVEIK |
| Hu004 VH1 | 11 | EVQLVQSGAEVKKPGSSVKVSCKASGYTFSDYNIHWVRQAPGQGLE |
| | | WMGYVKPNSGGTNYNQEFKDRVTITVDKSTSTAYMELSSLRSEDTA |
| | | VYYCARPHDYGSRTDYFDSWGQGTTVTVSS |
| Hu004 VL1 | 12 | DVQMTQSPSSLSASVGDRVTITCKASQNVHNDVAWYQQKPGKAPK |
| | | LLIYFASDRYTGVPSRFSGSGYGTDFTFTISSLQPEDIATYYCQQDYN |
| | | SPFTFGQGTKLEIK |
| Hu001 VH3 | 13 | EVQLVQSGAEVKKPGASVKVSCKASGYTFTSYWITWVRQRPGQGL |
| | | EWMGDIYPGSAITNYNDKFKNRVTITVDTSASTAYMELSSLRSEDTA |
| | | VYYCARHYINPYFYGMDYWGQGTTVTVSS |
| Hu001 VL3 | 14 | DTVMTQTPLSLSVTPGQPASISCKSSQSLFNSGNRENYLTWYQQKPG |
| | | QPPQLLIYWASTRESGVPDRFSGSGSGTDFTLKISRVEAEDVGVYYC |
| | | QNDYNYPLTFGQGTKLEIK |
| HcAb5-H5 | 15 | EVQLVESGGGLVQPGGSLRLSCAASGVTHSSYTVGWFRQAPGKERE |
| | | FVASITWNYGHTYYPDPEKGRFTISGDFAKNSVYLQMNSLRAEDTA |
| | | VYYCAAKLLGFGYADQGRYRYWGQGTTVTVSS |
| Guselkumab-VH | 16 | EVQLVQSGAEVKKPGESLKISCKGSGYSFSNYWIGWVRQMPGKGLE |
| | | WMGIIDPSNSYTRYSPSFQGQVTISADKSISTAYLQWSSLKASDTAM |
| | | YYCARWYYKPFDVWGQGTLVTVSS |
| Guselkumab-VL | 17 | QSVLTQPPSVSGAPGQRVTISCTGSSSNIGSGYDVHWYQQLPGTAPK |
| | | LLIYGNSKRPSGVPDRFSGSKSGTSASLAITGLQSEDEADYYCASWT |
| | | DGLSLVVFGGGTKLTVL |
| Risankizumab-VH | 18 | QVQLVQSGAEVKKPGSSVKVSCKASGYTFTDQTIHWMRQAPGQGL |
| | | EWIGYIYPRDDSPKYNENFKGKVTITADKSTSTAYMELSSLRSEDTA |
| | | VYYCAIPDRSGYAWFIYWGQGTLVTVSS |
| Risankizumab-VL | 19 | DIQMTQSPSSLSASVGDRVTITCKASRDVAIAVAWYQQKPGKVPKL |
| | | LIYWASTRHTGVPSRFSGSGSRTDFTLTISSLQPEDVADYFCHQYSSY |
| | | PFTFGSGTKLEIK |
| 37D5v1 VH | 20 | EVQLLESGGGLVQPGGSLRLSCAASGFTLDYLAIGWFRQAPGKGRE |
| | | GVSCVSSSGQYTYYADSVKGRFTISRDNSESTVYLQMNSLRPEDTAV |
| | | YYCATDPECYRVRGYYNGEYDYWGQGTLVTVSS |
| IgG1 Fc1 | 21 | EPKSCDKTHTCPPCPAPEAAGAPSVFLFPPKPKDTLMISRTPEVTCVV |
| | | VDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSR |
| | | EEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPG |
| IgG1 Fc2 | 22 | EPKSCDKTHTCPPCPAPEAAGGPSVFLFPPKPKDTLMISRTPEVTCVV |
| | | VDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSR |
| | | EEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPG |
| IgG1 Fc3 | 23 | EPKSCDKTHTCPPCPAPEAAGGPSVFLFPPKPKDTLYITREPEVTCVV |
| | | VDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSR |
| | | EEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPG |
| IgG1 Fc Knob 1 | 24 | EPKSCDKTHTCPPCPAPEAAGGPSVFLFPPKPKDTLMISRTPEVTCVV |
| | | VDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSR |
| | | EEMTKNQVSLWCLVKGFYPSDIAVEWESNGQPENNYKTTPPVLDSD |
| | | GSFFLYSKLTVDKSRWQQGNVFSCSVMHEALHNHYTQKSLSLSPG |
| IgG1 Fc Knob 2 | 25 | EPKSCDKTHTCPPCPAPEAAGGPSVFLFPPKPKDTLYITREPEVTCVV |
| | | VDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSR |
| | | EEMTKNQVSLWCLVKGFYPSDIAVEWESNGQPENNYKTTPPVLDSD |
| | | GSFFLYSKLTVDKSRWQQGNVFSCSVMHEALHNHYTQKSLSLSPG |
| IgG1 Fc Hole 1 | 26 | DKTHTCPPCPAPEAAGGPSVFLFPPKPKDTLMISRTPEVTCVVVDVS |
| | | HEDPEVKFNWYVDGVEVHNAKTKPREEQYNSTYRVVSVLTVLHQD |
| | | WLNGKEYKCKVSNKALPAPIEKTISKAKGQPREPQVYTLPPSREEMT |
| | | KNQVSLSCAVKGFYPSDIAVEWESNGQPENNYKTTPPVLDSDGSFFL |
| | | VSKLTVDKSRWQQGNVFSCSVMHEALHNHYTQKSLSLSPG |
| IgG1 Fc Hole 2 | 27 | DKTHTCPPCPAPEAAGGPSVFLFPPKPKDTLYITREPEVTCVVVDVSH |
| | | EDPEVKFNWYVDGVEVHNAKTKPREEQYNSTYRVVSVLTVLHQD |
| | | WLNGKEYKCKVSNKALPAPIEKTISKAKGQPREPQVYTLPPSREEMT |
| | | KNQVSLSCAVKGFYPSDIAVEWESNGQPENNYKTTPPVLDSDGSFFL |
| | | VSKLTVDKSRWQQGNVFSCSVMHEALHNHYTQKSLSLSPG |
| IgG1 CH1 | 28 | ASTKGPSVFPLAPSSKSTSGGTAALGCLVKDYFPEPVTVSWNSGALT |
| | | SGVHTFPAVLQSSGLYSLSSVVTVPSSSLGTQTYICNVNHKPSNTKV |
| | | DKRV |
| CL_κ | 29 | RTVAAPSVFIFPPSDEQLKSGTASVVCLLNNFYPREAKVQWKVDNA |
| | | LQSGNSQESVTEQDSKDSTYSLSSTLTLSKADYEKHKVYACEVTHQ |
| | | GLSSPVTKSFNRGEC |
| CL_λ | 30 | GQPKAAPSVTLFPPSSEELQANKATLVCLISDFYPGAVTVAWKADSS |
| | | PVKAGVETTTPSKQSNNKYAASSYLSLTPEQWKSHRSYSCQVTHEG |
| | | STVEKTVAPTECS |
| L1((G4S)₄) | 31 | GGGGSGGGGSGGGGSGGGGS |
| L2((G4S)₃) | 32 | GGGGSGGGGSGGGGS |
| HcAb7-H5c | 69 | EVQLVESGGGVVQPGRSLRLSCVASGYAYRMGWFRQTTGKEREGV |
| | | AVIYTGDAHTYYADSAKGRFTISQDNSKNTVYLQMNDLRAEDTAV |
| | | YYCAAGRGPMLRPLSSYGIEYWGQGTMVTVSS |
| HcAb8-H4a | 70 | EVQLVESGGGLVQPGGSLRLSCAASGDTSSDTLMGWFRQAPGKERE |
| | | GVAAIYTGGGSTYYADSAPGRFTISEDNAKNSVYLEMNSLRAEDTA |
| | | VYYCASPGRVGFFPSPLRPSNYKYWGQGTLVTVSS |
| PAb VH7 | 71 | EVQLVQSGAEVKKPGESLKISCKGSGYSFSNYWIGWVRQMPGKGLE |
| | | WMGIIDPSNTYSRYSPSFQGQVTISADKSISTAYLQWSSLKASDTAM |
| | | YYCARWYYKPFDVWGQGTLVTVSS |
| PAb VL8 | 72 | QSVLTQPPSVSGAPGQRVTISCSGTSSNIGSGYDVHWYQQLPGTAPK |
| | | LLIYGNSKRPAGVPDRFSGSKSGTSASLAITGLQSEDEADYYCASWT |
| | | DGLSLVVFGGGTKLTVL |
| PAb VH3 | 73 | EVQLVQSGAEVKKPGESLKISCKGSGYSFSNYWIGWVRQMPGKGLE |
| | | WMGIIDPSNSYVRYSPSFQGQVTISADKSISTAYLQWSSLKASDTAM |
| | | YYCARWYYKPFDVWGQGTLVTVSS |
| PAb VL5 | 74 | QSVLTQPPSVSGAPGQRVTISCSGTSSNIGSGYDVHWYQQLPGTAPK |
| | | LLIYGNSKRPSGVPDRFSGSKSGTSASLAITGLQSEDEADYYCASWT |
| | | DGLSLVVFGGGTKLTVL |

TABLE 7
Analysis of antibody CDR sequences (Kabat numbering)

| Sequence name | CDR1 | CDR2 | CDR3 |
|---|---|---|---|
| RVT3101.VL | RASQSVSSYLA | DASNRAT | QQRSNWPWT |
| | (SEQ ID NO: 33) | (SEQ ID NO: 34) | (SEQ ID NO: 35) |
| RVT3101.VH | YYGIS | WISTYNGNTHYARMLQG | ENYYGSGAYRGGMDV |
| | (SEQ ID NO: 36) | (SEQ ID NO: 37) | (SEQ ID NO: 38) |
| Hu001.VL3 | KSSQSLFNSGNRENYLT | WASTRES | QNDYNYPLT |
| | (SEQ ID NO: 39) | (SEQ ID NO: 40) | (SEQ ID NO: 41) |
| Hu001.VH3 | SYWIT | DIYPGSAITNYNDKFKN | HYINPYFYGMDY |
| | (SEQ ID NO: 42) | (SEQ ID NO: 43) | (SEQ ID NO: 44) |
| Hu004. VL1 | KASQNVHNDVA | FASDRYT | QQDYNSPFT |
| | (SEQ ID NO: 45) | (SEQ ID NO: 46) | (SEQ ID NO: 47) |
| Hu004.VH1 | DYNIH | YVKPNSGGTNYNQEFKD | PHDYGSRTDYFDS |
| | (SEQ ID NO: 48) | (SEQ ID NO: 49) | (SEQ ID NO: 50) |
| HcAb5-H5 | SYTVG | SITWNYGHTYYPDPEKG | KLLGFGYADQGRYRY |
| | (SEQ ID NO: 51) | (SEQ ID NO: 52) | (SEQ ID NO: 53) |
| Guselkumab-VH | NYWIG | IIDPSNSYTRYSPSFQG | WYYKPFDV |
| | (SEQ ID NO: 54) | (SEQ ID NO: 55) | (SEQ ID NO: 56) |
| Guselkumab-VL | TGSSSNIGSGYDVH | GNSKRPS | ASWTDGLSLVV |
| | (SEQ ID NO: 57) | (SEQ ID NO: 58) | (SEQ ID NO: 59) |
| Risankizumab- | DQTIH | YIYPRDDSPKYNENFKG | PDRSGYAWFIY |
| VH | (SEQ ID NO: 60) | (SEQ ID NO: 61) | (SEQ ID NO: 62) |
| Risankizumab- | KASRDVAIAVA | WASTRHT | HQYSSYPFT |
| VL | (SEQ ID NO: 63) | (SEQ ID NO: 64) | (SEQ ID NO: 65) |
| 37D5v1 VH | YLAIG | CVSSSGQYTYYADSVKG | DPECYRVRGYYNGEYDY |
| | (SEQ ID NO: 66) | (SEQ ID NO: 67) | (SEQ ID NO: 68) |
| HcAb7-H5c | MG | VIYTGDAHTYYADSAKG | GRGPMLRPLSSYGIEY |
| | (SEQ ID NO: 75) | (SEQ ID NO: 76) | (SEQ ID NO: 77) |
| HcAb8-H4a | DTLMG | AIYTGGGSTYYADSAPG | PGRVGFFPSPLRPSNYKY |
| | (SEQ ID NO: 78) | (SEQ ID NO: 79) | (SEQ ID NO: 80) |
| PAb VH7 | NYWIG | IIDPSNTYSRYSPSFQG | WYYKPFDV |
| | (SEQ ID NO: 54) | (SEQ ID NO: 81) | (SEQ ID NO: 56) |
| PAb VL8 | SGTSSNIGSGYDVH | GNSKRPA | ASWTDGLSLVV |
| | (SEQ ID NO: 82) | (SEQ ID NO: 83) | (SEQ ID NO: 59) |
| PAb VH3 | NYWIG | IIDPSNSYVRYSPSFQG | WYYKPFDV |
| | (SEQ ID NO: 54) | (SEQ ID NO: 84) | (SEQ ID NO: 56) |
| PAb VL5 | SGTSSNIGSGYDVH | GNSKRPS | ASWTDGLSLVV |
| | (SEQ ID NO: 85) | (SEQ ID NO: 58) | (SEQ ID NO: 59) |

### Example 3. Identification of Activity of Anti-TL1A×IL23p19 Bispecific Molecules

### 3.1 Assay on Binding Activity of Bispecific Molecules to Human TL1A-his Protein by ELISA

The human TL1A-his protein (prepared in Example 1.1) was diluted with PBS to a final concentration of 2 μg/mL, and then added to a 96-well ELISA plate at 50 μL/well. The plate was sealed with a plastic film and incubated at 4° C. overnight. The next day, the plate was washed twice with PBST, and a blocking buffer [PBS+5% (w/w) skimmed milk] was added for blocking at room temperature for 1 h. The plate was washed 3 times with PBST, and each test bispecific molecule or the control antibody (RVT3101) serially diluted 8-fold from 100 nM was added at 50 μL/well. After incubation at 37° C. for 1 h, the plate was washed 3 times with PBST. An HRP enzyme-labeled Goat anti human antibody (Jackson, Cat. No.: 109-035-098) was added. After incubation at 37° C. for 1 h, the plate was washed 5 times with PBST. A TMB substrate was added at 50 μL/well. After incubation at room temperature for 4 min, a stop solution (1.0 N HCl) was added at 50 μL/well. OD450 nm values were read using an ELISA plate reader (Multimode Plate Reader, EnSight, purchased from Perkin Elmer). Then, analysis was performed by software (GraphPad Prism9), data were fitted, and EC50 values were calculated. As shown in FIG. 2 and Table 8, the bispecific molecules of the present disclosure were all able to effectively bind to the human TL1A-his protein.

### 3.2 Assay on Blocking of Interaction Between Human TL1A and CHO-K1 Human DR3 Cells by Bispecific Molecules by FACS

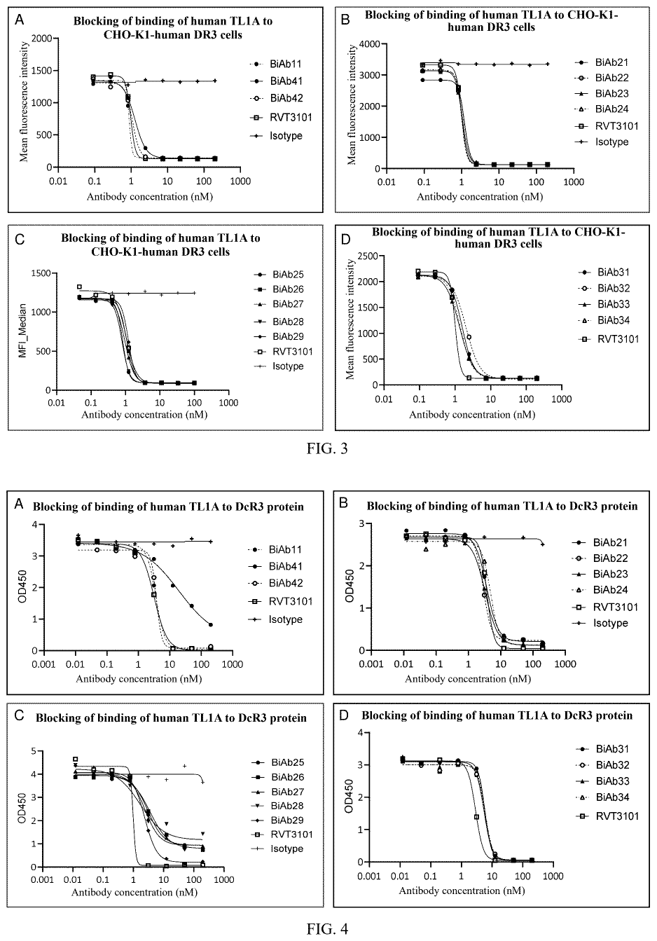

CHO-K1 human DR3 cells (prepared in Example 1.3) were expanded to 90% confluence in a T-75 cell culture flask, the medium was removed by pipetting, and the cells were washed twice with a PBS buffer, digested with trypsin, and washed twice with a PBS buffer after the digestion was stopped. After cell counting, the cells were diluted to 2×10⁶ cells/mL with a blocking buffer and added to a 96-well reaction plate at 50 L/well. Each test antibody, 3-fold diluted from 400 nM, was mixed with hTL1A-his (prepared in Example 1.1) diluted with PBS to 0.4 μg/mL at a ratio of 1:1, and the mixture was incubated at room temperature for 30 min. After incubation, 50 μL of the mixed solution was added to the plate, and the plate was incubated on ice for 1 h. After the plate was centrifuged and washed 3 times with a PBS buffer, an Alexa Fluor 647-labeled secondary antibody (GenScript, Cat. No.: A01802) was added at 50 μL/well, and the plate was incubated on ice for 1 h. After the plate was centrifuged and washed 5 times with a PBS buffer, the results were assayed and analyzed by a flow cytometer (FACS Canto™, purchased from BD Biosciences). Data analysis was performed by software (FlowJo) to obtain the mean fluorescence intensity (MFI) of the cells. Then, analysis was performed by software (GraphPad Prism9), data were fitted, and IC50 values were calculated. As shown in FIG. 3 and Table 8, the bispecific molecules of the present disclosure were all able to effectively block the binding of the TL1A protein to CHO-K1-human DR3 cells.

### 3.3 Assay on Inhibition of Interaction Between Human TL1A and DcR3 by Bispecific Molecules by ELISA

Human DcR3/TNFRSF6B, Fc Tag protein (Acro, Cat. No.: TNB-H5255) was diluted with PBS to a final concentration of 2 μg/mL, and then added to a 96-well ELISA plate at 50 μL/well. The plate was sealed with a plastic film and incubated at 4° C. overnight. The next day, the plate was washed twice with PBST, and a blocking buffer [PBS+5% (w/w) skimmed milk] was added for blocking at room temperature for 1 h. The plate was washed 3 times with PBST. Each test antibody serially diluted 4-fold from 400 nM was mixed with the 1 μg/mL biotinylated hTL1A protein (prepared in Example 1.1) at a ratio of 1:1, and the mixture was incubated for 30 min. After incubation, 50 μL of the mixed solution was added to the ELISA plate. After incubation at 37° C. for 1 h, the plate was washed 3 times with PBST. An HRP (horseradish peroxidase)-labeled secondary antibody (Sigma, Cat. No.: S2438) was added. After incubation at 37° C. for 1 h, the plate was washed 5 times with PBST. A TMB substrate was added at 50 L/well. After incubation at room temperature for 4 min, a stop solution (1.0 N HCl) was added at 50 μL/well. OD450 nm values were read using an ELISA plate reader (Multimode Plate Reader, EnSight, purchased from Perkin Elmer). Then, analysis was performed by software (GraphPad Prism9), data were fitted, and IC50 values were calculated. As shown in FIG. 4 and Table 8, the bispecific molecules of the present disclosure were all able to block the binding of TL1A to DcR3.

### 3.4 Assay on Inhibition of NFκB Activity in TF-1-NFκB-Luciferase Cells by Bispecific Molecules

The TF-1-NFκB-luciferase cells (prepared in Example 1.4) were expanded to the logarithmic growth phase in a T-75 cell culture flask, the medium supernatant was discarded by direct centrifugation, and the cell pellet was washed twice with PBS. The cells were adjusted to a density of 8×10⁵/mL with a medium [1640+2% (w/w) FBS], and added to a white transparent-bottom reaction plate (kanovo, Cat. No.: 62096) at 50 μL/well. The cells were starved in a cell incubator at 37° C. for 24 h. The human TL1A-his protein (prepared in Example 1.1) was diluted with a diluent [1640+2% (w/w) FBS] to a final concentration of 0.6 μg/mL, and added to each test antibody serially diluted 4-fold at a ratio of 1:1. The plate was pre-incubated at room temperature for 30 min. After pre-incubation, 50 μL of the mixed solution was added to the plate with the starved cells, and the plate was incubated in an incubator at 37° C. for 5 h. The assay was performed using a Nano-Light luciferase reporter gene detection kit (MeilunBio, Cat. No.: MA0521-2), the reagent was added at 50 μL/well, and the plate was read by an instrument PE EnSight microplate reader. The results were recorded. The experimental results showed that the bispecific molecules were all able to inhibit the activation of the downstream NFκB signaling pathway caused by the binding of human TL1A to its receptor (FIG. 5 and Table 8).

### 3.5 Assay on Inhibition of Apoptotic Activity of TF-1 Cells by Bispecific Molecules

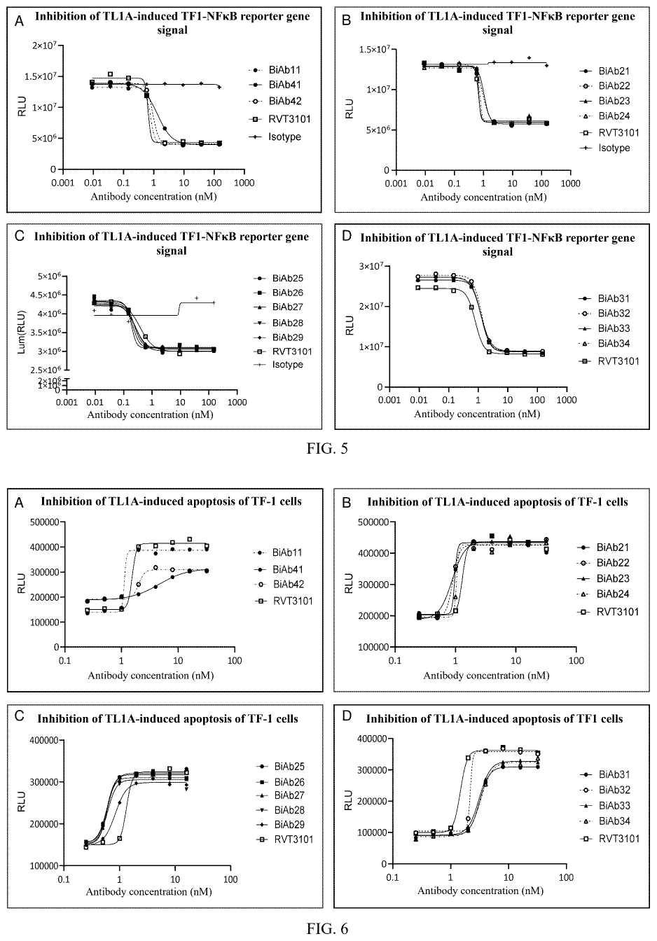

The total volume of the test system used in this example was 100 μL. A dilution of the human TL1A protein (prepared in Example 1.1) was prepared with an assay medium (RPMI1640+10% FBS+1× Penicillin/Streptomycin) to a final concentration of 100 ng/mL, and the dilution was added to a 96-well flat-bottom white plate (Corning, Cat. No.: 3917) at 20 μL/well. Each test antibody was diluted with an assay medium to a maximum final concentration of 32 nM. The dilution was performed 2-fold to obtain 8 concentration gradients. The dilution was added to a 96-well flat-bottom white plate at 20 μL/well, mixed with the TL1A protein, and incubated at room temperature for 30 min. TF-1 cells in the logarithmic growth phase were collected and resuspended in an assay medium, and CHX (MCE, Cat. No.: HY-12320) at a final concentration of 10 μg/mL was added. TF-1 cells were seeded into the 96-well flat-bottom white plate described above at 20000 cells/well, and the mixture was mixed well. The cell plate was incubated in an incubator at 37° C. with 5% CO₂ for 24 h. After 24 h, the cell plate was taken out from the incubator and allowed to equilibrate to room temperature. The cell viability detection reagent CellCounting-Lite 2.0 (Vazyme, Cat. No.: DD1101-03) equilibrated to room temperature was added at 100 μL/well, and the mixture was placed on a shaker, mixed well for 5 min, and left to stand at room temperature for 10 min. The fluorescence signals were measured using a microplate reader (PerkinElmer, model: Envision). The experimental results showed that the bispecific molecules were all able to inhibit the apoptotic activity of TF-1 cells (FIG. 6 and Table 8).

TABLE 8
Identification of blocking activity of bispecific molecules against binding of TL1A

| | | Human TL1A/DR3 | TL1A/DcR3 | TF-1 NFkB | TF-1 apoptotic |
|---|---|---|---|---|---|
| | Human TL1A | protein blocking | protein | reporter gene | activity inhibition |
| Sample | binding assay | assay | blocking assay | inhibition assay | assay |
| name | EC50(nM) | IC50(nM) | IC50(nM) | IC50(nM) | IC50(nM) |
| BiAb11 | 0.074 | 0.879 | 3.606 | 0.778 | 1.125 |
| BiAb41 | 0.108 | 1.271 | 19.67 | 1.319 | 4.608 |
| BiAb42 | 0.031 | 1.081 | 3.75 | 0.933 | 1.999 |
| RVT3101 | 0.047 | 0.969 | 3.166 | 0.651 | 1.547 |
| BiAb21 | 0.028 | 1.194 | 3.69 | 1.052 | 0.974 |
| BiAb22 | 0.041 | 1.003 | 2.896 | 0.948 | 0.898 |
| BiAb23 | 0.051 | 1.042 | 3.312 | 0.687 | 0.877 |
| BiAb24 | 0.052 | 1.119 | 4.887 | 0.757 | 1.044 |
| RVT3101 | 0.021 | 1.004 | 3.754 | 0.672 | 1.301 |
| BiAb31 | 0.063 | 1.571 | 5.848 | 1.261 | 3.168 |
| BiAb32 | 0.069 | 2.033 | 5.863 | 1.279 | 2.176 |
| BiAb33 | 0.065 | 1.33 | 5.454 | 1.216 | 3.118 |
| BiAb34 | 0.069 | 1.399 | 5.987 | 1.204 | 3.313 |
| RVT3101 | 0.04 | 1.001 | 2.907 | 0.789 | 1.471 |
| BiAb25 | 0.083 | 0.83 | 3.00 | 0.26 | 0.596 |
| BiAb26 | 0.086 | 1.09 | 3.57 | 0.20 | 0.594 |
| BiAb27 | 0.090 | 0.95 | 2.44 | 0.26 | 0.594 |
| BiAb28 | 0.086 | 0.79 | 1.71 | 0.19 | 0.609 |
| BiAb29 | 0.095 | 1.22 | 2.37 | 0.23 | 0.837 |
| RVT3101 | 0.030 | 1.04 | 0.99 | 0.33 | 1.33 |

Note:
Isotype in FIGS. 2-5 corresponding to Table 8 is a negative reference, embodied as N/A (not applicable).

3.6 Assay on Inhibition of IFN-γ Secretion from PBMCs Stimulated with Human TL1A in Cooperation with IL-12 and IL-18 by Bispecific Molecules

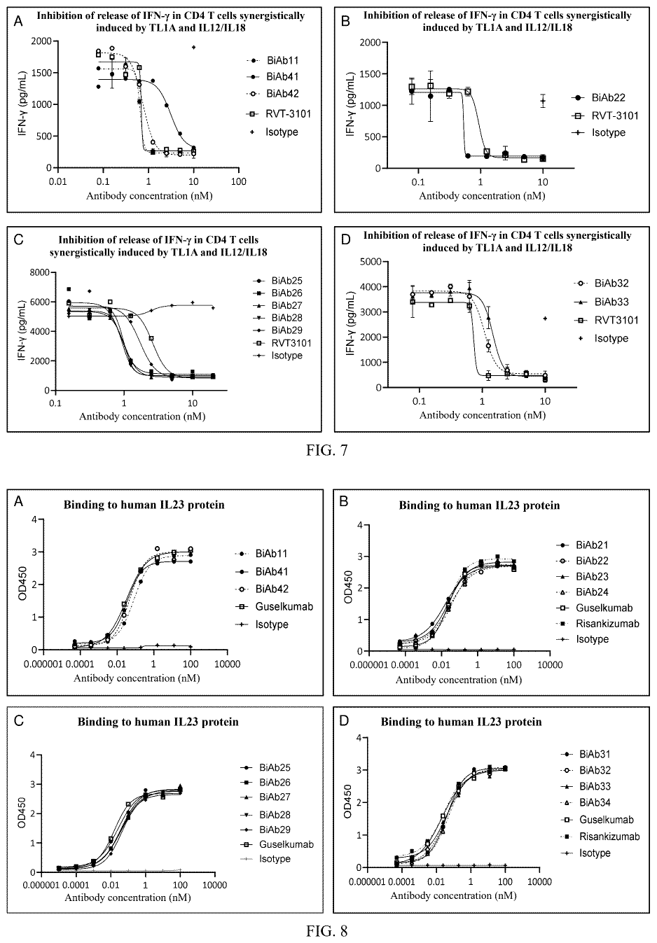

CD4⁺ T cells (Stemcell, Cat. No.: 17952) were isolated from peripheral blood mononuclear cells (PBMCs) of healthy human donors, adjusted to a cell density of 1 million/mL, and transferred to a 96-well U-bottom cell culture plate at 100 μL/well. An appropriate amount of recombinant human interleukin 18 (rhIL-18, R&D, Cat. No.: 9124-IL-050, working concentration: 50 ng/mL), recombinant human interleukin 12 (rhIL-12, Peprotech, Cat. No.: 200-12, working concentration: 2 ng/ml), and recombinant human TL1A (prepared in Example 1.1, working concentration: 50 ng/mL) were formulated, and the rhIL-18/rhIL-12/rhTL1A cytokine mixed solution was added at 50 μL/well to induce the activation of CD4⁺ T cells. Each test bispecific molecule or the control antibody was formulated with the corresponding working concentrations being 10 nM, 5 nM, 2.5 nM, 1.25 nM, 0.625 nM, 0.3125 nM, 0.15625 nM, and 0.078125 nM, and the corresponding test antibody was added at 50 μL/well. The mixture was mixed well and then cultured in a cell incubator at 37° C. with 5% CO₂ for 2 days. Then, the cell culture supernatant was collected, and the IFN-γ content in the supernatant was determined using a Human IFN-γ Precoated ELISA Kit (Dayou, Cat. No.: 1110003) according to the reagent instructions. As shown in FIG. 7, the bispecific molecules of the present disclosure were able to effectively inhibit IFN-γ secretion from PBMCs stimulated with human TL1A in cooperation with IL-12/IL-18.

### 3.7 Assay on Binding Activity of Bispecific Molecules to Human IL23-his Protein by ELISA

A 96-well ELISA plate was pre-coated with 5 μg/mL Streptavidin (purchased from Sigma, Cat. No.: S4762) diluted with PBS, incubated overnight at 4° C., washed twice with PBST, and blocked with a blocking buffer [PBS+5% (w/w) skimmed milk] at room temperature for 1 h. The human Biotin-hIL23-his protein (prepared in Example 1.2) was diluted with 1% (w/w) BSA-PBS to a final concentration of 2 μg/mL, and then added to a 96-well ELISA plate at 50 μL/well. After incubation at 37° C. for 1 h, the plate was washed 3 times with PBST, and each test antibody serially diluted 8-fold from 100 nM was added at 50 μL/well. After incubation at 37° C. for 1 h, the plate was washed 3 times with PBST. An HRP (horseradish peroxidase)-labeled secondary antibody (Jackson Immuno, Cat. No.: 109-035-098) was added. After incubation at 37° C. for 1 h, the plate was washed 5 times with PBST. A TMB substrate was added at 50 μL/well. After incubation at room temperature for 4 min, a stop solution (1.0 N HCl) was added at 50 μL/well. OD450 nm values were read using an ELISA plate reader (Multimode Plate Reader, EnSight, purchased from Perkin Elmer). Then, analysis was performed by software (GraphPad Prism10), data were fitted, and EC50 values were calculated. The experimental results showed that the bispecific molecules were all able to effectively bind to the human IL23 protein (FIG. 8 and Table 9).

### 3.8 Assay on Blocking of Interaction Between Human IL23 and IL23R Protein by Bispecific Molecules by ELISA

Human IL23R-hFc protein (purchased from Sino Biological, Cat. No.: 13840-H02H) was diluted with PBS to a final concentration of 2 μg/mL and then added to a 96-well ELISA plate at 50 μL/well. The plate was sealed with a plastic film and incubated at 4° C. overnight. The next day, the plate was washed twice with PBST, and a blocking buffer [PBS+5% (w/w) skimmed milk] was added for blocking at room temperature for 1 h. The plate was washed 3 times with PBST. Each test antibody serially diluted 4-fold from 400 nM was mixed with 0.2 μg/mL Biotin-hIL23-his (prepared in Example 1.2) at a ratio of 1:1, and the mixture was incubated for 30 min. After incubation, 50 μL of the mixed solution was added to the ELISA plate. After incubation at 37° C. for 1 h, the plate was washed 3 times with PBST. An HRP (horseradish peroxidase)-labeled secondary antibody (Sigma, Cat. No.: S2438) was added. After incubation at 37° C. for 1 h, the plate was washed 5 times with PBST. A TMB substrate was added at 50 μL/well. After incubation at room temperature for 4 min, a stop solution (1.0 N HCl) was added at 50 μL/well. OD450 nm values were read using an ELISA plate reader (Multimode Plate Reader, EnSight, purchased from Perkin Elmer). Analysis was performed by software (GraphPad Prism10), data were fitted, and IC50 values were calculated. As shown in FIG. 9, the bispecific molecules of the present disclosure were all able to effectively block the binding of the IL23 protein to the IL23R protein (Table 9).

### 3.9 Assay on Blocking of Interaction Between Human IL23 and CHO-K1-Human IL23R Cells by Bispecific Molecules by FACS

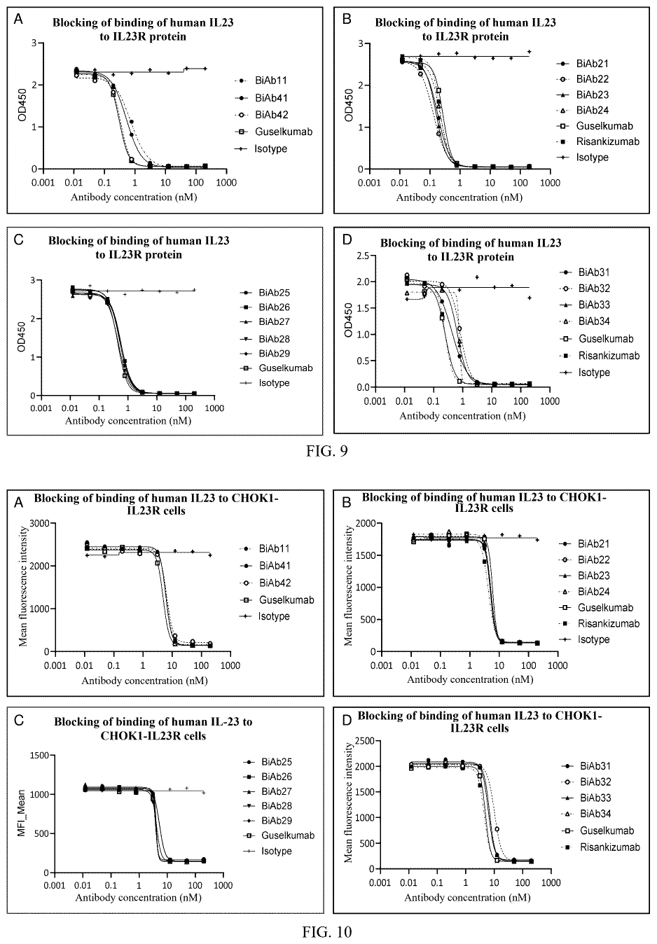

Adherent cells, namely CHO-K1-human IL23R cells (constructed in Example 1.5), were expanded to 90% confluence in a cell culture flask, and the medium was removed by pipetting. The cells were washed twice with a PBS buffer. The cells were then digested with trypsin and washed twice with a PBS buffer after the digestion was stopped. After cell counting, the cell pellet was resuspended to 1×10⁶ cells/mL with a blocking buffer [PBS+2% (w/w) FBS], and the cells were added to a 96-well FACS reaction plate at 50 μL/well. Each test antibody serially diluted 4-fold from 400 nM was mixed with 2 μg/mL Biotin-hIL23-his (prepared in Example 1.2) at a ratio of 1:1, and the mixture was incubated for 30 min. The mixed solution was added to the plate at 50 μL/well, and the plate was incubated at 4° C. for 1 h. The plate was centrifuged and washed 3 times with a PBS buffer, an APC-labeled secondary antibody (BioLegend, Cat. No.: 405243) was added at 50 μL/well, and the plate was incubated on ice for 1 h. After the plate was centrifuged and washed 3 times with a PBS buffer, the cells were resuspended, and the results were assayed and analyzed by FACS (FACS Canto™, purchased from BD Biosciences). Data analysis was performed by software (FlowJo) to obtain the mean fluorescence intensity (MFI) of the cells. Analysis was performed by software (GraphPad Prism10), data were fitted, and IC50 values were calculated. The experimental results showed that the bispecific molecules were all able to effectively block the binding of human IL23 to CHO-K1-human IL23R cells (FIG. 10 and Table 9).

### 3.10 Blocking of 1123-Activated STAT3 Signaling Pathway Downstream of HEK293-IL23R by Bispecific Molecules

IL23 is a heterodimeric cytokine consisting of IL-23A (P19) and IL-12B (P40). When IL-23 binds to the heterodimeric receptor complex consisting of IL-12RB1/IL-23R, the JAK-STAT signaling pathway is activated, leading to a proinflammatory response. The most important signaling pathway is JAK2-STAT3. In this example, based on the test principle described above, the cell strain 293-IL23 Res (Novoprotein, Cat. No.: XCC05-1) expressing IL12RB1, IL23R, and STAT3 reporter genes was selected to test the blocking effect of the antibodies on the IL-23-activated STAT3 signaling pathway. On day 0, 293-IL23 Res cells in the exponential growth phase were collected, digested, counted, and centrifuged, and the supernatant was discarded. The cells were resuspended in a DMEM (Gibco, Cat. No.: 10569-010) complete medium containing 10% fetal bovine serum (Excell, Cat. No.: FSP500) and 1% penicillin/streptomycin (Gibco, Cat. No.: 15140-122) and counted. The cell density was adjusted to 5×10⁵/mL with the complete medium, and the cells were added to a 96-well white transparent flat-bottom cell culture plate (Costar, Cat. No.: 3599) at 100 μL/well and cultured overnight.

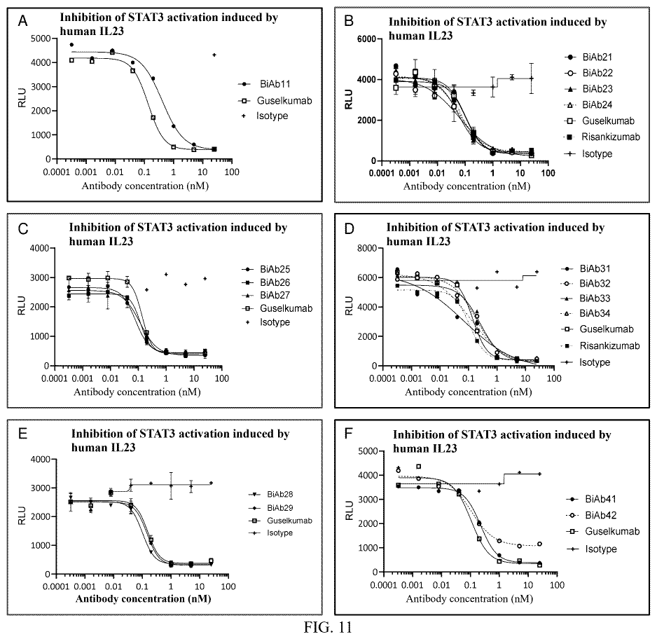

On day 1, the human IL23 protein was formulated to a final concentration of 0.05 μg/mL in a DMEM (Gibco, Cat. No.: 10569-010) complete medium containing 2% fetal bovine serum (Excell, Cat. No.: FSP500) and 1% penicillin/streptomycin (Gibco, Cat. No.: 15140-122), and each test antibody was diluted with the complete medium (initial concentration: 50 nM, 5-fold dilution, 8 concentration gradients in total). The IL-23 protein dilution was then mixed with the serially diluted antibody in equal volumes, and the mixture was incubated at room temperature for 30 min. The supernatant was discarded after the cells were cultured overnight, and then the co-incubation substance of the antibody and the IL-23 protein was added to the plate at 100 μL/well. The plate was incubated in an incubator at 37° C. for 6 h. Assay was performed using a KeyTec® enhanced luciferase reporter gene detection kit (VKEY-BIO, Cat. No.: A2000100N), and 100 μL of detection reagent was added to each well. The plate was shaken and mixed well for 8 min, and chemiluminescence signals were detected by a PerkinElmer EnVision multi-mode microplate detector. Data analysis was performed using the Graphpad Prism software. The experimental results showed that the bispecific molecules were all able to effectively block the IL23-activated STAT3 signaling pathway downstream of HEK293-IL23R (FIG. 11 and Table 9).

### 3.11 Inhibition of IL23-Induced IL17 Factor Release in Mouse Splenocytes by Bispecific Molecules

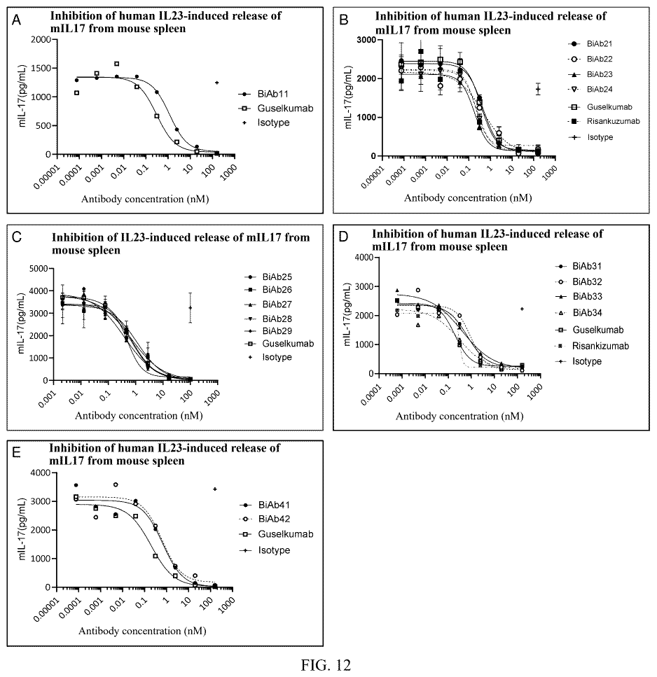

After C57BL/6 mice (Shanghai Lingchang Biotechnology) were euthanized, the spleen was collected, ground through a 70 μm cell sieve, and resuspended in an RPMI1640 medium (Gibco, Cat. No.: 22400-089). The mixture was centrifuged at 300 g for 8 min, and the supernatant was discarded. 4 mL of red blood cell lysis buffer (Gibco, Cat. No.: A10492-01) was added to lyse the red blood cells, and a pre-cooled DPBS buffer was added to terminate the reaction. The mixture was centrifuged at 300 g for 8 min, and the supernatant was discarded. The splenocytes were resuspended in an RPMI-1640 complete medium (containing 10% fetal bovine serum, 1% penicillin/streptomycin, 50 μM β-mercaptoethanol, and 20 ng/mL mouse IL-2) and counted. The cell density was adjusted to 5×10⁶/mL. The cells were added to a 96-well U-bottom cell plate (Costar, Cat. No.: 3799) at 100 μL/well. The serially diluted antibody (initial concentration: 640 nM, 8-fold dilution, 8 concentration gradients in total) was premixed with the human IL-23 protein (final concentration: 20 ng/ml) in equal volumes for 30 min, and then 100 μL of the incubated substance was added to a cell plate and incubated at 37° C. After 3 days, the culture supernatant was collected and assayed for mouse IL-17A content using a mouse IL-17 ELISA kit (Valukine, Cat. No.: VAL610), and data analysis was performed using the Graphpad Prism software. As shown in FIG. 12 and Table 9, the bispecific molecules of the present disclosure were all able to effectively inhibit the IL23-induced IL17 factor release in mouse splenocytes.

TABLE 9
Identification of blocking activity of bispecific molecules against binding of IL23

| | | Human | | | |
|---|---|---|---|---|---|
| | Human | IL23/ | Human | | |
| | IL23 | IL23R | IL23/CHOK1- | HEK293- | |
| | binding | protein | IL23R cell | IL23R-STAT3 | Mouse spleen |
| | assay | blocking | blocking | reporter gene | mIL 17 release |
| | EC50 | assay | assay | inhibition assay | inhibition assay |
| Sample name | (nM) | IC50(nM) | IC50(nM) | IC50(nM) | IC50(nM) |
| BiAb11 | 0.082 | 0.719 | 6.251 | 0.411 | 1.167 |
| BiAb41 | 0.034 | 0.526 | 6.037 | 0.221 | 0.668 |
| BiAb42 | 0.05 | 0.34 | 6.462 | 0.106 | 0.613 |
| Guselkumab | 0.032 | 0.304 | 4.873 | Corresponding | Corresponding |
| | | | | BiAb11: 0.135 | BiAb11: 0.198 |
| | | | | Corresponding | Corresponding |
| | | | | BiAb41-42: 0.106 | BiAb41-42: 0.303 |
| BiAb21 | 0.017 | 0.182 | 5.024 | 0.101 | 0.436 |
| BiAb22 | 0.03 | 0.132 | 4.92 | 0.063 | 0.483 |
| BiAb23 | 0.026 | 0.162 | 5.241 | 0.059 | 0.183 |
| BiAb24 | 0.038 | 0.22 | 5.131 | 0.063 | 0.24 |
| Guselkumab | 0.023 | 0.27 | 5.982 | 0.106 | 0.369 |
| Risankizumab | 0.025 | 0.222 | 4.304 | 0.073 | 0.204 |
| BiAb31 | 0.042 | 0.427 | 6.176 | 0.071 | 0.681 |
| BiAb32 | 0.041 | 0.83 | 10.22 | 0.186 | 0.966 |
| BiAb33 | 0.043 | 0.661 | 6.83 | 0.259 | 0.408 |
| BiAb34 | 0.051 | 0.772 | 6.805 | 0.119 | 0.314 |
| Guselkumab | 0.022 | 0.251 | 4.903 | 0.14 | 0.192 |
| Risankizumab | 0.029 | 0.249 | 4.426 | 0.111 | 0.254 |
| BiAb25 | 0.027 | 0.52 | 3.93 | 0.079 | 0.707 |
| BiAb26 | 0.040 | 0.54 | 5.36 | 0.120 | 1.152 |
| BiAb27 | 0.039 | 0.58 | 5.25 | 0.117 | 0.386 |
| BiAb28 | 0.041 | 0.46 | 4.17 | 0.137 | 0.498 |
| BiAb29 | 0.043 | 0.58 | 4.08 | 0.156 | 0.663 |
| Guselkumab | 0.016 | 0.44 | 3.86 | Corresponding | 0.654 |
| | | | | BiAb25-27: 0.137 | |
| | | | | Corresponding | |
| | | | | BiAb28-29: 0.155 | |

Note:
1. Isotype in FIGs. 8-12 corresponding to Table 9 is a negative reference, embodied as N/A (not applicable);
2. In FIGs. 11-12, molecules BiAb11 and BiAb41-42 were assayed separately, and in FIG. 11, BiAb25-27 and BiAb28-29 were assayed separately, so the corresponding values of the positive reference Guselkumab used are shown separately.

### 3.12 Assay on Simultaneous Binding of Bispecific Molecules to Human TL1A and Human IL23 by ELISA

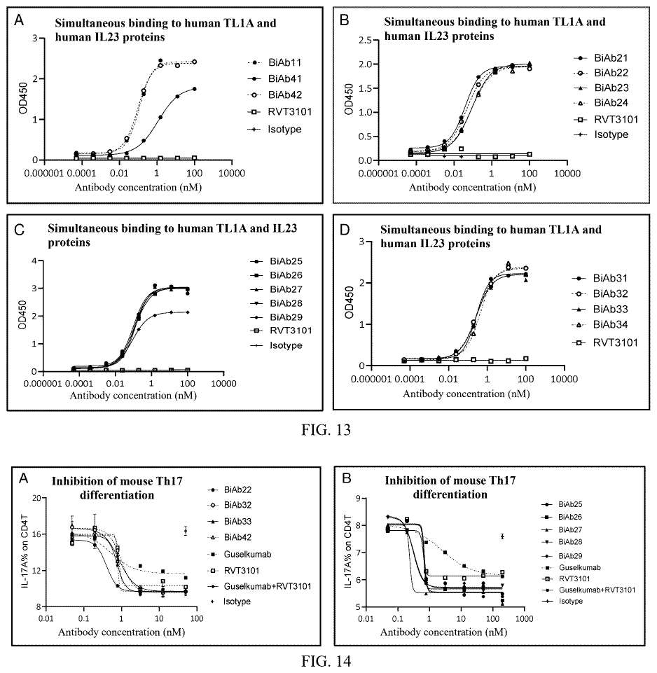

A 96-well ELISA plate was coated with the human TL1A-his protein (prepared in Example 1.1) diluted to 2 μg/mL with PBS, incubated overnight at 4° C., washed twice with PBST, and blocked with a blocking buffer [PBS+5% (w/w) skimmed milk] at room temperature for 1 h. Each test antibody was subjected to an 8-fold dilution from 100 nM, and added to the ELISA plate at 50 μL/well. The plate was incubated at 37° C. for 1 h. The plate was washed 3 times with PBST. The human Biotin-hIL23-his protein (prepared in Example 1.2) was diluted with 1% (w/w) BSA-PBS to a final concentration of 2 μg/mL, and then added to a 96-well ELISA plate at 50 μL/well. The plate was incubated at 37° C. for 1 h and then washed 3 times with PBST. An HRP (horseradish peroxidase)-labeled detection antibody (Sigma, Cat. No.: S2438) was added. After incubation at 37° C. for 1 h, the plate was washed 5 times with PBST. A TMB substrate was added at 50 μL/well. After incubation at room temperature for 4 min, a stop solution (1.0 N HCl) was added at 50 μL/well. OD450 nm values were read using an ELISA plate reader (Multimode Plate Reader, EnSight, purchased from Perkin Elmer). Then, analysis was performed by software (GraphPad Prism10), data were fitted, and EC50 values were calculated. As shown in FIG. 13 and Table 10, the experimental results showed that the bispecific molecules were all able to bind to the human TL1A protein and the IL23 protein simultaneously.

TABLE 10
Identification of simultaneous binding of bispecific molecules to TL1A and IL23 proteins

| Sample | EC50 | Emax | Sample | EC50 | Emax | Sample | EC50 | Emax | Sample | EC50 | Emax |
|---|---|---|---|---|---|---|---|---|---|---|---|
| BiAb41 | 1.145 | 1.801 | BiAb21 | 0.038 | 1.956 | BiAb25 | 0.097 | 3.024 | BiAb31 | 0.269 | 2.223 |
| BiAb42 | 0.096 | 2.387 | BiAb22 | 0.047 | 1.942 | BiAb26 | 0.118 | 3.021 | BiAb32 | 0.298 | 2.386 |
| BiAb11 | 0.116 | 2.425 | BiAb23 | 0.086 | 2.004 | BiAb27 | 0.127 | 3.041 | BiAb33 | 0.268 | 2.205 |
| RVT3101 | N/A | 0.045 | BiAb24 | 0.084 | 1.951 | BiAb28 | 0.107 | 2.998 | BiAb34 | 0.428 | 2.351 |
| Isotype | N/A | 0.050 | RVT3101 | N/A | 0.138 | BiAb29 | 0.087 | 2.140 | RVT3101 | N/A | 0.245 |
| / | / | / | Isotype | N/A | 0.095 | RVT3101 | N/A | 0.066 | / | / | / |
| / | / | / | / | / | / | Isotype | N/A | 0.071 | / | / | / |

Note:
Isotype in FIG. 13 corresponding to Table 10 is a negative reference, and the EC50 value is embodied as N/A (not applicable).

### 3.13 Assay on Inhibition of TL1A- and IL23-Induced Th17 Differentiation by Bispecific Molecules

After mice (C57/BL/6, female, 6-8 weeks old, Beijing Vitalstar) were euthanized, the spleen was collected and ground to form a splenocyte suspension. The mouse CD4+ T cells were sorted from the mouse splenocytes using a mouse CD4+ T cell sorting kit (Miltenyi, Cat. No.: 130-090-862). The mouse CD4+ T cells were adjusted to a density of 0.75×10⁶/mL using an RPMI medium (Gibco, Cat. No.: 11875093) containing 10% FBS (Gibco, Cat. No.: A56696701), and an anti-mouse CD28 antibody (invitrogen, Cat. No.: 16-0281-86) at a final concentration of 5 μg/mL, 20 ng/ml mouse IL6 (Novoprotein, Cat. No.: CG93), and 1 ng/mL mouse TGF-β (Acrobiosystems, Cat. No.: TG1-M5218) were added. The mixture was plated for later use. A 96-well flat-bottom plate (Thermo, Cat. No.: 260860) was coated with 1 μg/mL anti-mouse CD3 antibody (Invitrogen, Cat. No.: 16-0031-85) at 100 L/well and incubated overnight at 4° C. The liquid in the original well plate was discarded. The plate was washed twice with DPBS (Hyclone, Cat. No.: SH30256.01), and the suspension of cells and stimulating factor described above was added at 200 μL/well for plating. The plate was incubated in an incubator at 37° C. with 5% CO₂ for 6-7 days. A half-medium change was performed every 3-4 days. After the incubation, the mouse Th17 cell differentiation proportion was determined.

Induced differentiated mouse Th17 cells were collected, resuspended in an RPMI1640 medium, adjusted to a concentration of 2×10⁶ cells/mL, and plated at 100 μL/well. A 4× concentration stimulation factor mixture (mouse TGF-β: 4 ng/mL, mouse IL6: 80 ng/mL, human IL23 (prepared in Example 1.2): 400 ng/mL, and mouse TL1A (prepared in Example 1.1): 400 ng/ml) was formulated and then plated at 50 μL/well. An initial test antibody at a 4× concentration of 200 nM was formulated, and then serially diluted 4-fold to obtain a total of 7 concentrations. The mixture was then plated at 50 μL/well. After being mixed well, the mixture was incubated in an incubator at 37° C. with 5% CO₂ for 3 days, and the mouse Th17 cell proportion was determined. Before the assay, an eBioscience™ cell stimulation reagent (eBioscience, Cat. No.: 00-4975-03) was added to stimulate the generation of factors and block their secretion extracellularly, and the cells were incubated for 4 h. The cells were collected and centrifuged, and then a diluted live/dead dye LIVE/DEAD™ Fixable Near-IR (Thermo, Cat. No.: L10119) was added at 100 μL/well for staining at 4° C. for 20 min. The cells were washed once with a staining buffer (DPBS containing 2% FBS). A fixation solution (BD Pharmingen, Cat. No.: 554714) was added at 100 μL/well for fixation at 4° C. for 20 min. The plate was washed once with a 1× Perm buffer (BD Pharmingen, Cat. No.: 554714). Anti-mouse IFN-γ PE-Cy7 (BD Pharmingen, Cat. No.: 557649) and anti-mouse IL17A BV421 (BD Pharmingen, Cat. No.: 563354) dilutions were formulated using a 1× Perm buffer at 100 μL/well, and the cells were stained at 4° C. for 30 min. The plate was washed twice with a 1× Perm buffer. The percentage of IL17A-positive cells (IL17A⁺%) in viable cells was determined by a flow cytometer (Thermo, Attune N×T).

The results are shown in FIG. 14 and Table 11. The bispecific molecules were able to reduce the mouse Th17 differentiation proportion in a dose-dependent manner.

TABLE 11
Inhibition of TL1A- and IL23-induced Th17 differentiation by
bispecific molecules

| Sample name | IC50(nM) | AUC | Imax |
|---|---|---|---|
| BiAb22 | 0.3979 | 90.12 | 9.682 |
| BiAb32 | 0.9848 | 91.29 | 9.594 |
| BiAb33 | 0.8575 | 99.64 | 9.704 |
| BiAb42 | 0.7867 | 93.48 | 9.72 |
| Guselkumab | 0.5289 | 188.5 | 11.66 |
| RVT3101 | 0.8517 | 131.8 | 10.33 |
| Gus + RVT3101 | 0.764 | 92.58 | 9.637 |
| BiAb25 | 0.672 | 84.3 | 5.727 |
| BiAb26 | 0.6811 | 84.4 | 5.66 |
| BiAb27 | 0.2309 | 84.5 | 5.627 |
| BiAb28 | 0.3156 | 84.7 | 5.701 |
| BiAb29 | 0.2964 | 133.9 | 5.777 |
| Guselkumab | 2.046 | 126.5 | 6.277 |
| RVT3101 | 0.6601 | 105.8 | 6.093 |
| Gus + RVT3101 | 0.6849 | 80.5 | 5.59 |

Note:
Isotype in FIG. 14 corresponding to Table 11 is a negative reference, embodied as N/A (not applicable).

### Example 4. Pharmacodynamic Study of Bispecific Molecules in Inflammatory Bowel Disease (IBD) Mice Models Induced by Cytokine Combination

Female BALB/c mice, 6-8 weeks old, were selected and fed a piroxicam diet for 4 days and randomized to different groups (G1-G8) according to body weight. G2-G8 groups were intraperitoneally injected once daily with a mixture of cytokines including mouse IL12, mouse IL18, human IL23, and human TL1A. Body weights of mice were measured daily during the experiment. On the day of model induction, mice were grouped according to body weight, and the test drug and drug combination were intraperitoneally injected on the day of group assignment (day 0) and day 3. The dose of each group was equimolar. Grouping settings are shown in Table 12.

TABLE 12
Dosing Regimens for the In Vivo Pharmacodynamic Study of Bispecific
Molecules in a Cytokine Combination-Induced IBD Mouse Model

| | | Dosage | Administration route and |
|---|---|---|---|
| Group | Drug | (mg/kg, mpk ) | frequency |
| G1 | / | / | / |
| G2 | Vehicle (PBS) | / | Day 0 and Day 3, |
| G3 | RVT3101 | 3.05 | intraperitoneal injection |
| G4 | HcAb7-H5c | 1.64 | |
| G5 | PAb H7L8 | 3 | |
| G6 | HcAb7-H5c + | HcAb7-H5c: 1.64 | |
| | PAb H7L8 | PAb H7L8: 3 | |
| G7 | BiAb25 | 3.60 | |
| G8 | BiAb29 | 3.60 | |

Note:
RVT3101 and HcAb7-H5c are anti-TL1A antibodies (with an IgG1 subtype Fc); PAb H7L8 is an anti-IL23 (P19 subunit) (IL23p19) antibody (with an IgG1 subtype Fc).

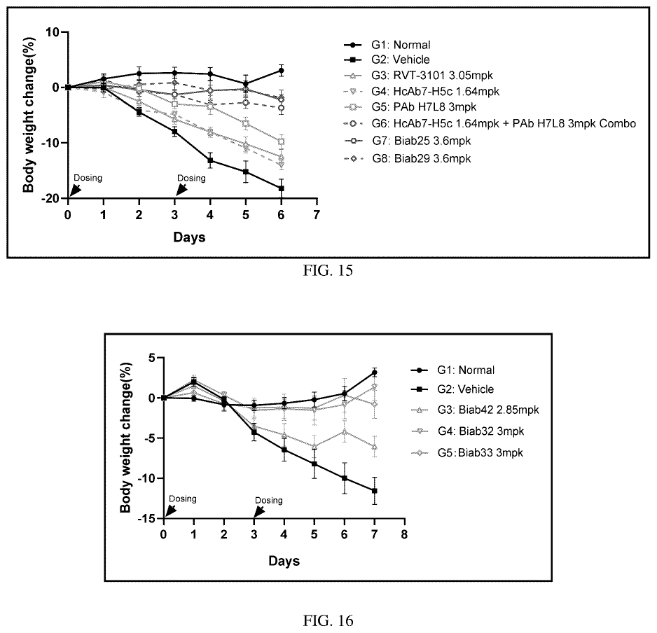

As shown in FIG. 15, compared with the model control group (G2), body weight loss in mice was significantly ameliorated in the anti-TL1A antibody monotherapy groups (G3, G4) and the anti-IL-23 antibody monotherapy group (G5). These data indicate that monoclonal antibodies only targeting TL1A and IL-23 individually exerted therapeutic effects in mice having this model of inflammatory bowel disease.

The combination therapy group (G6) displayed superior pharmacodynamic activity in improving body weight relative to the monotherapy groups (G3, G4, G5), supporting the synergistic efficacy of the combined administration of anti-IL23 antibody and anti-TL1A antibody treatment for inflammatory bowel disease.

Notably, treatment with the bispecific molecules (G7, G8) resulted in further enhanced efficacy compared with the combination therapy group (G6). These findings suggest that combining anti-IL-23 and anti-TL1A specificities into a single bispecific molecule can achieve an additional synergistic effect beyond co-administration of the two parental antibodies, thereby further potentiating pharmacodynamic activity.

### Example 5. Pharmacodynamic Study of Bispecific Molecules in Inflammatory Bowel Disease (IBD) Mice Models Induced by Cytokine Combination

Female BALB/c mice, 6-8 weeks old, were selected and fed a piroxicam diet for 4 days and randomized to different groups (G1-G5) according to body weight. G2-G5 groups were intraperitoneally injected once daily with a mixture of cytokines including mouse IL12, mouse IL18, human IL23, and human TL1A. Body weights of mice were measured daily during the experiment. On the day of model induction, mice were grouped according to body weight, and the test drug were intraperitoneally injected on the day of group assignment (day 0) and day 3. The dose of each group was equimolar. Grouping settings are shown in Table 13.

TABLE 13
Dosing Regimens for the In Vivo Pharmacodynamic Study of Bispecific
Molecules in a Cytokine Combination-Induced IBD Mouse Model

| | | Dosage | Administration route and |
|---|---|---|---|
| Group | Drug | (mg/kg, mpk ) | frequency |
| G1 | / | / | / |
| G2 | Vehicle (PBS) | / | Day 0 and Day 3, |
| G3 | BiAb42 | 2.85 | intraperitoneal injection |
| G4 | BiAb32 | 3 | |
| G5 | BiAb33 | 3 | |

As shown in FIG. 16, compared with the G2 model control group, treatment with the bispecific molecules (G3, G4, G5) significantly alleviated weight loss in mice with inflammatory bowel disease.

The teachings of all patents, published applications, and references cited herein are incorporated by reference in their entirety.

While exemplary examples have been specifically shown and described, it will be understood by those skilled in the art that various changes in forms and details may be made therein without departing from the scope of the embodiments encompassed by the appended claims.

1. A multispecific molecule binding to TL1A and IL23, comprising an antigen-binding domain specifically binding to TL1A, and an antigen-binding domain specifically binding to IL23, wherein the antigen-binding domain specifically binding to TL1A comprises:
(1) an HCDR1, an HCDR2, and an HCDR3 of the sequence set forth in SEQ ID NO: 70; or
(2) an HCDR1, an HCDR2, and an HCDR3 of the sequence set forth in SEQ ID NO: 69; or
(3) an HCDR1, an HCDR2, and an HCDR3 of the sequence set forth in SEQ ID NO: 15; and
wherein the HCDR1-3 and/or the LCDR1-3 are determined according to the Kabat, Chothia, or IMGT scheme.

2. The multispecific molecule according to claim 1, wherein the antigen-binding domain specifically binding to TL1A comprises a heavy chain variable region (VH) sequence having the sequence set forth in any one of SEQ ID NO: 70, 69, or 15, or a VH sequence having at least 97% identity compared thereto.

3. The multispecific molecule according to claim 1, wherein the antigen-binding domain specifically binding to IL23 comprises:
(1) an HCDR1, an HCDR2, and an HCDR3 of the sequence set forth in SEQ ID NO: 16, and an LCDR1, an LCDR2, and an LCDR3 of the sequence set forth in SEQ ID NO: 17; or
(2) an HCDR1, an HCDR2, and an HCDR3 of the sequence set forth in SEQ ID NO: 71, and an LCDR1, an LCDR2, and an LCDR3 of the sequence set forth in SEQ ID NO: 72; or
(3) an HCDR1, an HCDR2, and an HCDR3 of the sequence set forth in SEQ ID NO: 18, and an LCDR1, an LCDR2, and an LCDR3 of the sequence set forth in SEQ ID NO: 19; or
(4) an HCDR1, an HCDR2, and an HCDR3 of the sequence set forth in SEQ ID NO: 73, and an LCDR1, an LCDR2, and an LCDR3 of the sequence set forth in SEQ ID NO: 74; and
wherein the HCDR1-3 and/or the LCDR1-3 are determined according to the Kabat, Chothia, or IMGT scheme.

4. The multispecific molecule according to claim 1, wherein the antigen-binding domain specifically binding to IL23 comprises a heavy chain variable region (VH) sequence having the sequence set forth in any one of SEQ ID NO: 16, 71, 18, or 73, or a VH sequence having at least 98% identity compared thereto.

5. The multispecific molecule according to claim 1, wherein the antigen-binding domain specifically binding to IL23 comprises a light chain variable region (VL) sequence having the sequence set forth in any one of SEQ ID NO: 17, 72, 19 or 74, or a VL having at least 98% identity compared thereto.

6. The multispecific molecule according to claim 1, wherein the molecule comprises an IgG antibody and a VHH structure, wherein the VHH structure can be located at the N-terminus or C-terminus of a heavy chain or a light chain of the IgG antibody.

7. A bispecific molecule that binds specifically to TL1A and IL23, comprising:
(1) (i) an IL-23-targeting moiety, comprising: (a) a VH region having the sequence set forth in SEQ ID NO:16, linked to a CH1 region having the sequence set forth in SEQ ID NO:28; and (b) a VL region having the sequence set forth in SEQ ID NO:17, linked to a CL region having the sequence set forth in SEQ ID NO:30;
(ii) an Fc region formed by polypeptides having the sequence set forth in SEQ ID NO: 23;
(iii) a TL1A-targeting moiety having the sequence set forth in SEQ ID NO:70; and
(iv) a linker region between the Fc region and the TL1A-targeting moiety, having the sequence set forth in SEQ ID NO:32; or
(2) (i) an IL-23-targeting moiety, comprising: (a) a VH region having the sequence set forth in SEQ ID NO:71, linked to a CH1 region having the sequence set forth in SEQ ID NO:28; and (b) a VL region having the sequence set forth in SEQ ID NO:72, linked to a CL region having the sequence set forth in SEQ ID NO:30;
(ii) an Fc region formed by polypeptides having the sequence set forth in SEQ ID NO: 23;
(iii) a TL1A-targeting moiety having the sequence set forth in SEQ ID NO:69; and
(iv) a linker region between the Fc region and the TL1A-targeting moiety, having the sequence set forth in SEQ ID NO:32; or
(3) (i) an IL-23-targeting moiety, comprising: (a) a VH region having the sequence set forth in SEQ ID NO:73, linked to a CH1 region having the sequence set forth in SEQ ID NO:28; and (b) a VL region having the sequence set forth in SEQ ID NO:74, linked to a CL region having the sequence set forth in SEQ ID NO:30;
(ii) an Fc region formed by polypeptides having the sequence set forth in SEQ ID NO: 23;
(iii) a TL1A-targeting moiety having the sequence set forth in SEQ ID NO:69; and
(iv) a linker region between the Fc region and the TL1A-targeting moiety, having the sequence set forth in SEQ ID NO:32; or
(4) (i) an IL-23-targeting moiety, comprising: (a) a VH region having the sequence set forth in SEQ ID NO:73, linked to a CH1 region having the sequence set forth in SEQ ID NO:28; and (b) a VL region having the sequence set forth in SEQ ID NO:72, linked to a CL region having the sequence set forth in SEQ ID NO:30;
(ii) an Fc region formed by polypeptides having the sequence set forth in SEQ ID NO: 23;
(iii) a TL1A-targeting moiety having the sequence set forth in SEQ ID NO:69; and
(iv) a linker region between the Fc region and the TL1A-targeting moiety, having the sequence set forth in SEQ ID NO:32; or
(5) (i) an IL-23-targeting moiety, comprising: (a) a VH region having the sequence set forth in SEQ ID NO:16, linked to a CH1 region having the sequence set forth in SEQ ID NO:28; and (b) a VL region having the sequence set forth in SEQ ID NO:17, linked to a CL region having the sequence set forth in SEQ ID NO:30;
(ii) an Fc region formed by polypeptides having the sequence set forth in SEQ ID NO: 23;
(iii) a TL1A-targeting moiety having the sequence set forth in SEQ ID NO:69; and
(iv) a linker region between the Fc region and the TL1A-targeting moiety, having the sequence set forth in SEQ ID NO:32; or
(6) (i) an IL-23-targeting moiety, comprising: (a) a VH region having the sequence set forth in SEQ ID NO:16, linked to a CH1 region having the sequence set forth in SEQ ID NO:28; and (b) a VL region having the sequence set forth in SEQ ID NO:17, linked to a CL region having the sequence set forth in SEQ ID NO:30;
(ii) an Fc region formed by polypeptides having the sequence set forth in SEQ ID NO: 22;
(iii) a TL1A-targeting moiety having the sequence set forth in SEQ ID NO:15; and
(iv) a linker region between the Fc region and the TL1A-targeting moiety, having the sequence set forth in SEQ ID NO:32; or
(7) (i) an IL-23-targeting moiety, comprising: (a) a VH region having the sequence set forth in SEQ ID NO:16, linked to a CH1 region having the sequence set forth in SEQ ID NO:28; and (b) a VL region having the sequence set forth in SEQ ID NO:17, linked to a CL region having the sequence set forth in SEQ ID NO:30;
(ii) an Fc region formed by polypeptides having the sequence set forth in SEQ ID NO: 23;
(iii) a TL1A-targeting moiety having the sequence set forth in SEQ ID NO:15; and
(iv) a linker region between the Fc region and the TL1A-targeting moiety, having the sequence set forth in SEQ ID NO:32; or
(8) (i) an IL-23-targeting moiety, comprising: (a) a VH region having the sequence set forth in SEQ ID NO:18, linked to a CH1 region having the sequence set forth in SEQ ID NO:28; and (b) a VL region having the sequence set forth in SEQ ID NO:19, linked to a CL region having the sequence set forth in SEQ ID NO:29;
(ii) an Fc region formed by polypeptides having the sequence set forth in SEQ ID NO: 22;
(iii) a TL1A-targeting moiety having the sequence set forth in SEQ ID NO:15; and
(iv) a linker region between the Fc region and the TL1A-targeting moiety, having the sequence set forth in SEQ ID NO:32; or
(9) (i) an IL-23-targeting moiety, comprising: (a) a VH region having the sequence set forth in SEQ ID NO:18, linked to a CH1 region having the sequence set forth in SEQ ID NO:28; and (b) a VL region having the sequence set forth in SEQ ID NO:19, linked to a CL region having the sequence set forth in SEQ ID NO:29;
(ii) an Fc region formed by polypeptides having the sequence set forth in SEQ ID NO: 23;
(iii) a TL1A-targeting moiety having the sequence set forth in SEQ ID NO:15; and
(iv) a linker region between the Fc region and the TL1A-targeting moiety, having the sequence set forth in SEQ ID NO:32.

8. A bispecific molecule that binds specifically to TL1A and IL23, comprising:
(1) (i) a first polypeptide and a second polypeptide, each comprising from N-terminus to C-terminus:
(a) a VH region having the sequence set forth in SEQ ID NO:16;
(b) a CH1 region having the sequence set forth in SEQ ID NO:28;
(c) an Fc region sequence having the sequence set forth in SEQ ID NO:23;
(d) a linker sequence having the sequence set forth in SEQ ID NO:32; and
(e) a VHH region having the sequence set forth in SEQ ID NO:70; and
(ii) a third polypeptide and a fourth polypeptide, each comprising from N-terminus to C-terminus:
(a) a VL region having the amino acid sequence of SEQ ID NO:17; and
(b) a CL region having the amino acid sequence of SEQ ID NO:30; or
(2) (i) a first polypeptide and a second polypeptide, each comprising from N-terminus to C-terminus:
(a) a VH region having the sequence set forth in SEQ ID NO:71;
(b) a CH1 region having the sequence set forth in SEQ ID NO:28;
(c) an Fc region sequence having the sequence set forth in SEQ ID NO:23;
(d) a linker sequence having the sequence set forth in SEQ ID NO:32; and
(e) a VHH region having the sequence set forth in SEQ ID NO:69; and
(ii) a third polypeptide and a fourth polypeptide, each comprising from N-terminus to C-terminus:
(a) a VL region having the amino acid sequence of SEQ ID NO:72; and
(b) a CL region having the amino acid sequence of SEQ ID NO:30; or
(3) (i) a first polypeptide and a second polypeptide, each comprising from N-terminus to C-terminus:
(a) a VH region having the sequence set forth in SEQ ID NO:73;
(b) a CH1 region having the sequence set forth in SEQ ID NO:28;
(c) an Fc region sequence having the sequence set forth in SEQ ID NO:23;
(d) a linker sequence having the sequence set forth in SEQ ID NO:32; and
(e) a VHH region having the sequence set forth in SEQ ID NO:69; and
(ii) a third polypeptide and a fourth polypeptide, each comprising from N-terminus to C-terminus:
(a) a VL region having the amino acid sequence of SEQ ID NO:74; and
(b) a CL region having the amino acid sequence of SEQ ID NO:30; or
(4) (i) a first polypeptide and a second polypeptide, each comprising from N-terminus to C-terminus:
(a) a VH region having the sequence set forth in SEQ ID NO:73;
(b) a CH1 region having the sequence set forth in SEQ ID NO:28;
(c) an Fc region sequence having the sequence set forth in SEQ ID NO:23;
(d) a linker sequence having the sequence set forth in SEQ ID NO:32; and
(e) a VHH region having the sequence set forth in SEQ ID NO:69; and
(ii) a third polypeptide and a fourth polypeptide, each comprising from N-terminus to C-terminus:
(a) a VL region having the amino acid sequence of SEQ ID NO:72; and
(b) a CL region having the amino acid sequence of SEQ ID NO:30; or
(5) (i) a first polypeptide and a second polypeptide, each comprising from N-terminus to C-terminus:
(a) a VH region having the sequence set forth in SEQ ID NO:16;
(b) a CH1 region having the sequence set forth in SEQ ID NO:28;
(c) an Fc region sequence having the sequence set forth in SEQ ID NO:23;
(d) a linker sequence having the sequence set forth in SEQ ID NO:32; and
(e) a VHH region having the sequence set forth in SEQ ID NO:69; and
(ii) a third polypeptide and a fourth polypeptide, each comprising from N-terminus to C-terminus:
(a) a VL region having the amino acid sequence of SEQ ID NO:17; and
(b) a CL region having the amino acid sequence of SEQ ID NO:30; or
(6) (i) a first polypeptide and a second polypeptide, each comprising from N-terminus to C-terminus:
(a) a VH region having the sequence set forth in SEQ ID NO:16;
(b) a CH1 region having the sequence set forth in SEQ ID NO:28;
(c) an Fc region sequence having the sequence set forth in SEQ ID NO:22;
(d) a linker sequence having the sequence set forth in SEQ ID NO:32; and
(e) a VHH region having the sequence set forth in SEQ ID NO:15; and
(ii) a third polypeptide and a fourth polypeptide, each comprising from N-terminus to C-terminus:
(a) a VL region having the amino acid sequence of SEQ ID NO:17; and
(b) a CL region having the amino acid sequence of SEQ ID NO:30; or
(7) (i) a first polypeptide and a second polypeptide, each comprising from N-terminus to C-terminus:
(a) a VH region having the sequence set forth in SEQ ID NO:16;
(b) a CH1 region having the sequence set forth in SEQ ID NO:28;
(c) an Fc region sequence having the sequence set forth in SEQ ID NO:23;
(d) a linker sequence having the sequence set forth in SEQ ID NO:32; and
(e) a VHH region having the sequence set forth in SEQ ID NO:15; and
(ii) a third polypeptide and a fourth polypeptide, each comprising from N-terminus to C-terminus:
(a) a VL region having the amino acid sequence of SEQ ID NO:17; and
(b) a CL region having the amino acid sequence of SEQ ID NO:30; or
(8) (i) a first polypeptide and a second polypeptide, each comprising from N-terminus to C-terminus:
(a) a VH region having the sequence set forth in SEQ ID NO:18;
(b) a CH1 region having the sequence set forth in SEQ ID NO:28;
(c) an Fc region sequence having the sequence set forth in SEQ ID NO:22;
(d) a linker sequence having the sequence set forth in SEQ ID NO:32; and
(e) a VHH region having the sequence set forth in SEQ ID NO:15; and
(ii) a third polypeptide and a fourth polypeptide, each comprising from N-terminus to C-terminus:
(a) a VL region having the amino acid sequence of SEQ ID NO:19; and
(b) a CL region having the amino acid sequence of SEQ ID NO:29; or
(9) (i) a first polypeptide and a second polypeptide, each comprising from N-terminus to C-terminus:
(a) a VH region having the sequence set forth in SEQ ID NO:18;
(b) a CH1 region having the sequence set forth in SEQ ID NO:28;
(c) an Fc region sequence having the sequence set forth in SEQ ID NO:23;
(d) a linker sequence having the sequence set forth in SEQ ID NO:32; and
(e) a VHH region having the sequence set forth in SEQ ID NO:15; and
(ii) a third polypeptide and a fourth polypeptide, each comprising from N-terminus to C-terminus:
(a) a VL region having the amino acid sequence of SEQ ID NO:19; and
(b) a CL region having the amino acid sequence of SEQ ID NO:29.

9. An isolated nucleic acid, encoding the multispecific molecule according to claim 1.

10. A vector, comprising the nucleic acid according to claim 9.

11. A cell, comprising the isolated nucleic acid of claim 9.

12. A method for preparing the multispecific molecule according to claim 1, comprising: (1) culturing the cell according to claim 11 and/or (2) isolating a molecule expressed by the cell.

13. A pharmaceutical composition, comprising the multispecific molecule of claim 1.

14. A method for treating an immune-mediated inflammatory disease (IMID), comprising administering to a subject an effective amount of a pharmaceutical composition comprising the molecule according to claim 1, wherein, the immune-mediated inflammatory disease (IMID) is selected from one or more of the group consisting of an autoimmune disease inflammatory bowel disease, psoriasis, rheumatoid arthritis, psoriatic arthritis, arteriosclerosis, and uveitis.

15. The pharmaceutical composition of claim 13, wherein the composition further comprises, an additional therapeutic agent.


SEQUENCE LISTING (86 sequences)

SEQ ID NO: 1 - protein, 180 aa, Homo sapiens
LKGQEFAPSHQQVYAPLRADGDKPRAHLTVVRQTPTQHFKNQFPALHWEHELGLAFTKNRMNYTNKFLLIPESGDYFIYSQVTFRGMTSECSEIRQAGRPNKPDSITVVITKVTDSYPEPTQLLMGTKSVCEVGSNWFQPIYLGAMFSLQEGDKLMVNVSDISLVDYTKEDKTFFGAFLL

SEQ ID NO: 2 - protein, 192 aa, Mus musculus
AGQLRVPGKDCMLRAITEERSEPSPQQVYSPPRGKPRAHLTIKKQTPAPHLKNQLSALHWEHDLGMAFTKNGMKYINKSLVIPESGDYFIYSQITFRGTTSVCGDISRGRRPNKPDSITVVITKVADSYPEPARLLTGSKSVCEISNNWFQSLYLGAMFSLEEGDRLMVNVSDISLVDYTKEDKTFFGAFLL

SEQ ID NO: 3 - protein, 170 aa, Homo sapiens
RAVPGGSSPAWTQCQQLSQKLCTLAWSAHPLVGHMDLREEGDEETTNDVPHIQCGDGCDPQGLRDNSQFCLQRIHQGLIFYEKLLGSDIFTGEPSLLPDSPVGQLHASLLGLSQLLQPEGHHWETQQIPSLSPSQPWQRLLLRFKILRSLQAFVAVAARVFAHGAATLSP

SEQ ID NO: 4 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 5 - protein, 175 aa, Mus musculus
VPRSSSPDWAQCQQLSRNLCMLAWNAHAPAGHMNLLREEEDEETKNNVPRIQCEDGCDPQGLKDNSQFCLQRIRQGLAFYKHLLDSDIFKGEPALLPDSPMEQLHTSLLGLSQLLQPEDHPRETQQMPSLSSSQQWQRPLLRSKILRSLQAFLAIAARVFAHGAATLTEPLVPTA

SEQ ID NO: 6 - protein, 313 aa, Mus musculus
MWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRS

SEQ ID NO: 7 - protein, 417 aa, Homo sapiens
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 8 - protein, 629 aa, Homo sapiens
MNQVTIQWDAVIALYILFSWCHGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 9 - protein, 124 aa, synthetic construct
QVQLVQSGAEVKKPGASVKVSCKASGYDFTYYGISWVRQAPGQGLEWMGWISTYNGNTHYARMLQGRVTMTTDTSTRTAYMELRSLRSDDTAVYYCARENYYGSGAYRGGMDVWGQGTTVTVSS

SEQ ID NO: 10 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPWTFGQGTKVEIK

SEQ ID NO: 11 - protein, 122 aa, synthetic construct
EVQLVQSGAEVKKPGSSVKVSCKASGYTFSDYNIHWVRQAPGQGLEWMGYVKPNSGGTNYNQEFKDRVTITVDKSTSTAYMELSSLRSEDTAVYYCARPHDYGSRTDYFDSWGQGTTVTVSS

SEQ ID NO: 12 - protein, 107 aa, synthetic construct
DVQMTQSPSSLSASVGDRVTITCKASQNVHNDVAWYQQKPGKAPKLLIYFASDRYTGVPSRFSGSGYGTDFTFTISSLQPEDIATYYCQQDYNSPFTFGQGTKLEIK

SEQ ID NO: 13 - protein, 121 aa, synthetic construct
EVQLVQSGAEVKKPGASVKVSCKASGYTFTSYWITWVRQRPGQGLEWMGDIYPGSAITNYNDKFKNRVTITVDTSASTAYMELSSLRSEDTAVYYCARHYINPYFYGMDYWGQGTTVTVSS

SEQ ID NO: 14 - protein, 113 aa, synthetic construct
DTVMTQTPLSLSVTPGQPASISCKSSQSLFNSGNRENYLTWYQQKPGQPPQLLIYWASTRESGVPDRFSGSGSGTDFTLKISRVEAEDVGVYYCQNDYNYPLTFGQGTKLEIK

SEQ ID NO: 15 - protein, 124 aa, synthetic construct
EVQLVESGGGLVQPGGSLRLSCAASGVTHSSYTVGWFRQAPGKEREFVASITWNYGHTYYPDPEKGRFTISGDFAKNSVYLQMNSLRAEDTAVYYCAAKLLGFGYADQGRYRYWGQGTTVTVSS

SEQ ID NO: 16 - protein, 117 aa, synthetic construct
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSS

SEQ ID NO: 17 - protein, 111 aa, synthetic construct
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVL

SEQ ID NO: 18 - protein, 120 aa, synthetic construct
QVQLVQSGAEVKKPGSSVKVSCKASGYTFTDQTIHWMRQAPGQGLEWIGYIYPRDDSPKYNENFKGKVTITADKSTSTAYMELSSLRSEDTAVYYCAIPDRSGYAWFIYWGQGTLVTVSS

SEQ ID NO: 19 - protein, 107 aa, synthetic construct
DIQMTQSPSSLSASVGDRVTITCKASRDVAIAVAWYQQKPGKVPKLLIYWASTRHTGVPSRFSGSGSRTDFTLTISSLQPEDVADYFCHQYSSYPFTFGSGTKLEIK

SEQ ID NO: 20 - protein, 126 aa, synthetic construct
EVQLLESGGGLVQPGGSLRLSCAASGFTLDYLAIGWFRQAPGKGREGVSCVSSSGQYTYYADSVKGRFTISRDNSESTVYLQMNSLRPEDTAVYYCATDPECYRVRGYYNGEYDYWGQGTLVTVSS

SEQ ID NO: 21 - protein, 231 aa, synthetic construct
EPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 231 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 23 - protein, 231 aa, synthetic construct
EPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 231 aa, synthetic construct
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 25 - protein, 231 aa, synthetic construct
EPKSCDKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 27 - protein, 226 aa, synthetic construct
DKTHTCPPCPAPEAAGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 28 - protein, 98 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 29 - protein, 107 aa, synthetic construct [sequence omitted]

SEQ ID NO: 30 - protein, 106 aa, synthetic construct
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 31 - protein, 20 aa, synthetic construct
GGGGSGGGGSGGGGSGGGGS

SEQ ID NO: 32 - protein, 15 aa, synthetic construct
GGGGSGGGGSGGGGS

SEQ ID NO: 33 - protein, 11 aa, synthetic construct
RASQSVSSYLA

SEQ ID NO: 34 - protein, 7 aa, synthetic construct
DASNRAT

SEQ ID NO: 35 - protein, 9 aa, synthetic construct
QQRSNWPWT

SEQ ID NO: 36 - protein, 5 aa, synthetic construct
YYGIS

SEQ ID NO: 37 - protein, 17 aa, synthetic construct
WISTYNGNTHYARMLQG

SEQ ID NO: 38 - protein, 15 aa, synthetic construct
ENYYGSGAYRGGMDV

SEQ ID NO: 39 - protein, 17 aa, synthetic construct
KSSQSLFNSGNRENYLT

SEQ ID NO: 40 - protein, 7 aa, synthetic construct
WASTRES

SEQ ID NO: 41 - protein, 9 aa, synthetic construct
QNDYNYPLT

SEQ ID NO: 42 - protein, 5 aa, synthetic construct
SYWIT

SEQ ID NO: 43 - protein, 17 aa, synthetic construct
DIYPGSAITNYNDKFKN

SEQ ID NO: 44 - protein, 12 aa, synthetic construct
HYINPYFYGMDY

SEQ ID NO: 45 - protein, 11 aa, synthetic construct
KASQNVHNDVA

SEQ ID NO: 46 - protein, 7 aa, synthetic construct
FASDRYT

SEQ ID NO: 47 - protein, 9 aa, synthetic construct
QQDYNSPFT

SEQ ID NO: 48 - protein, 5 aa, synthetic construct
DYNIH

SEQ ID NO: 49 - protein, 17 aa, synthetic construct
YVKPNSGGTNYNQEFKD

SEQ ID NO: 50 - protein, 13 aa, synthetic construct
PHDYGSRTDYFDS

SEQ ID NO: 51 - protein, 5 aa, synthetic construct
SYTVG

SEQ ID NO: 52 - protein, 17 aa, synthetic construct
SITWNYGHTYYPDPEKG

SEQ ID NO: 53 - protein, 15 aa, synthetic construct
KLLGFGYADQGRYRY

SEQ ID NO: 54 - protein, 5 aa, synthetic construct
NYWIG

SEQ ID NO: 55 - protein, 17 aa, synthetic construct
IIDPSNSYTRYSPSFQG

SEQ ID NO: 56 - protein, 8 aa, synthetic construct
WYYKPFDV

SEQ ID NO: 57 - protein, 14 aa, synthetic construct
TGSSSNIGSGYDVH

SEQ ID NO: 58 - protein, 7 aa, synthetic construct
GNSKRPS

SEQ ID NO: 59 - protein, 11 aa, synthetic construct
ASWTDGLSLVV

SEQ ID NO: 60 - protein, 5 aa, synthetic construct
DQTIH

SEQ ID NO: 61 - protein, 17 aa, synthetic construct
YIYPRDDSPKYNENFKG

SEQ ID NO: 62 - protein, 11 aa, synthetic construct
PDRSGYAWFIY

SEQ ID NO: 63 - protein, 11 aa, synthetic construct
KASRDVAIAVA

SEQ ID NO: 64 - protein, 7 aa, synthetic construct
WASTRHT

SEQ ID NO: 65 - protein, 9 aa, synthetic construct
HQYSSYPFT

SEQ ID NO: 66 - protein, 5 aa, synthetic construct
YLAIG

SEQ ID NO: 67 - protein, 17 aa, synthetic construct
CVSSSGQYTYYADSVKG

SEQ ID NO: 68 - protein, 17 aa, synthetic construct
DPECYRVRGYYNGEYDY

SEQ ID NO: 69 - protein, 122 aa, synthetic construct
EVQLVESGGGVVQPGRSLRLSCVASGYAYRMGWFRQTTGKEREGVAVIYTGDAHTYYADSAKGRFTISQDNSKNTVYLQMNDLRAEDTAVYYCAAGRGPMLRPLSSYGIEYWGQGTMVTVSS

SEQ ID NO: 70 - protein, 127 aa, synthetic construct
EVQLVESGGGLVQPGGSLRLSCAASGDTSSDTLMGWFRQAPGKEREGVAAIYTGGGSTYYADSAPGRFTISEDNAKNSVYLEMNSLRAEDTAVYYCASPGRVGFFPSPLRPSNYKYWGQGTLVTVSS

SEQ ID NO: 71 - protein, 117 aa, synthetic construct
EVQLVQSGAEVKKPGESLKISCKGSGYSFSNYWIGWVRQMPGKGLEWMGIIDPSNTYSRYSPSFQGQVTISADKSISTAYLQWSSLKASDTAMYYCARWYYKPFDVWGQGTLVTVSS

SEQ ID NO: 72 - protein, 111 aa, synthetic construct
QSVLTQPPSVSGAPGQRVTISCSGTSSNIGSGYDVHWYQQLPGTAPKLLIYGNSKRPAGVPDRFSGSKSGTSASLAITGLQSEDEADYYCASWTDGLSLVVFGGGTKLTVL

SEQ ID NO: 73 - protein, 117 aa, synthetic construct
EVQLVQSGAEVKKPGESLKISCKGSGYSFSNYWIGWVRQMPGKGLEWMGIIDPSNSYVRYSPSFQGQVTISADKSISTAYLQWSSLKASDTAMYYCARWYYKPFDVWGQGTLVTVSS

SEQ ID NO: 74 - protein, 111 aa, synthetic construct
QSVLTQPPSVSGAPGQRVTISCSGTSSNIGSGYDVHWYQQLPGTAPKLLIYGNSKRPSGVPDRFSGSKSGTSASLAITGLQSEDEADYYCASWTDGLSLVVFGGGTKLTVL

SEQ ID NO: 75 - skipped

SEQ ID NO: 76 - protein, 17 aa, synthetic construct
VIYTGDAHTYYADSAKG

SEQ ID NO: 77 - protein, 16 aa, synthetic construct
GRGPMLRPLSSYGIEY

SEQ ID NO: 78 - protein, 5 aa, synthetic construct
DTLMG

SEQ ID NO: 79 - protein, 17 aa, synthetic construct
AIYTGGGSTYYADSAPG

SEQ ID NO: 80 - protein, 18 aa, synthetic construct
PGRVGFFPSPLRPSNYKY

SEQ ID NO: 81 - protein, 17 aa, synthetic construct
IIDPSNTYSRYSPSFQG

SEQ ID NO: 82 - protein, 14 aa, synthetic construct
SGTSSNIGSGYDVH

SEQ ID NO: 83 - protein, 7 aa, synthetic construct
GNSKRPA

SEQ ID NO: 84 - protein, 17 aa, synthetic construct
IIDPSNSYVRYSPSFQG

SEQ ID NO: 85 - protein, 14 aa, synthetic construct
SGTSSNIGSGYDVH

SEQ ID NO: 86 - protein, 5 aa, synthetic construct
GGGGS